SEQUENCE LISTING (11 sequences)

SEQ ID NO: 1 - protein, 528 aa, synthetic construct
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 533 aa, synthetic construct
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 3 - protein, 503 aa, synthetic construct
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 4 - protein, 506 aa, synthetic construct
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 5 - protein, 754 aa, synthetic construct
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 534 aa, synthetic construct
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 7 - DNA, 1669 bp, synthetic construct
  source 1..1669: mol_type = other DNA
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 8 - DNA, 1671 bp, synthetic construct
  source 1..1671: mol_type = other DNA
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 9 - DNA, 1259 bp, synthetic construct
  source 1..1259: mol_type = other DNA
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcaggaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 10 - protein, 420 aa, synthetic construct
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 11 - protein, 487 aa, synthetic construct
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

(19) United States
(12) Patent Application Publication
(10) Pub. No.: US 2026/0158119 A1
(43) Pub. Date: Jun. 11, 2026

(54) INTESTINAL ALKALINE PHOSPHATASES AND METHODS OF USE IN INHIBITING LIVER FIBROSIS

(71) Applicant: The General Hospital Corporation, Boston, MA (US)

(72) Inventor: Richard A. Hodin, Newton, MA (US)

(21) Appl. No.: 19/409,450

(22) Filed: Dec. 4, 2025

Related U.S. Application Data
(63) Continuation of application No. 18/018,394, now abandoned.
(60) Provisional application No. 63/059,781, filed on Jul. 31, 2020.

(51) Int. Cl. A61K 38/46; A61P 1/16

(52) U.S. Cl. CPC A61K 38/465; A61P 1/16; C12Y 301/03001

(57) ABSTRACT

The present disclosure relates, inter alia, to therapeutic alkaline phosphatases for the treatment of liver fibrosis.

### CROSS-REFERENCE TO RELATED APPLICATIONS

This application is a continuation of U.S. patent application Ser. No. 18/018,394, filed on Jan. 27, 2023, which is a U.S. National Phase Application under 35 U.S.C. § 371 of International Patent Application No. PCT/US2021/043691, filed on Jul. 29, 2021, which claims the benefit of U.S. Provisional Application No. 63/059,781, filed on Jul. 31, 2020, the contents of which is hereby incorporated by reference in its entirety.

### SEQUENCE LISTING

This application contains a Sequence Listing that has been submitted electronically as an XML file named “29539-0551002_SL_ST26.XML.” The XML file, created on Dec. 4, 2025, is 19,376 bytes in size. The material in the XML file is hereby incorporated by reference in its entirety.

### TECHNICAL FIELD

The present disclosure relates, inter alia, to therapeutic alkaline phosphatases, and uses thereof, for the treatment of liver fibrosis.

### BACKGROUND

Fibrosis is a common sequela in many chronic liver diseases, including hepatitis, benign or malignant bile duct obstruction or alcoholic liver disease. In recent years, an important relationship between liver pathology and the gastrointestinal tract has come to light (Ohtani et al., Hepatol Commun. 2019; 3:456-70). In particular, the pathophysiologic development of liver fibrosis has been linked to an impaired GI tract barrier. The intestinal barrier sits at the interface between the host and a microbial population that is the source of important mediators of intestinal and hepatic inflammation (Wang Y et al., Mol Med Rep. 2014; 9:2352-6; Murphy et al., Curr Opin Clin Nutr Metab Care. 2015; 18:515-20).

The two most commonly used animal models of liver fibrosis are induced chemically (carbon tetrachloride; CCl₄) or by bile duct ligation (BDL). CCl₄ causes hepatocyte damage, necrosis, inflammation, and fibrosis after 4 weeks of challenge and over 8 weeks causes cirrhosis. In contrast, BDL stimulates the proliferation of biliary epithelial cells and oval cells causing proliferating bile ductules with accompanying portal inflammation and fibrosis. Previous studies have reported that decreased tight junction protein expression and increased gut permeability occurs during the early stages of both common bile duct ligation (CBDL) and Carbon Tetrachloride-4 (CCl4) induced liver fibrosis murine models, although the models reflect different models of the human disease.

Further, intestinal bacterial overgrowth is often associated with liver fibrosis (Wiest et al., J Hepatol. 2014; 60:197-209); the increased burden of gut bacteria and pathogen associated molecular patterns (PAMPs) allow for translocation from the gut into the liver parenchyma via the portal venous system (Wiest et al., J Hepatol. 2014; 60:197-209). Interestingly, the depletion of intestinal bacteria by a cocktail of antibiotics or blockade of the lipopolysaccharides (LPS)-TLR4 pathway rescues liver fibrosis in murine models (Seki et al., Nat Med. 2007; 13:1324-32), thereby implicating the importance of the microbiome and microbial products in the development of this disease.

Intestinal alkaline phosphatase (IAP) is one subtype of the alkaline phosphatase family and is produced exclusively in the intestine with highest expression in the duodenum (Bilski et al., Mediators Inflamm. 2017; 2017:9074601). Endogenous IAP levels have been shown to be lower in gut-associated inflammatory conditions such as inflammatory bowel disease and diabetes mellitus (Tuin et al., Gut. 2009; 58:379-87; Ramasamy et al., Inflamm Bowel Dis. 2011; 17:532-42; Hamarneh et al., Dig Dis Sci. 2017). IAP levels have not been previously assessed in the context of liver fibrosis.

IAP is known to detoxify bacterial LPS by removing a phosphate group from its lipid A moiety, thus preventing recognition by its receptor TLR4 (Soares et al., Hepatol Int. 2010; 4:659-72). In addition, IAP maintains the gut barrier by up-regulating intestinal tight junction protein expression (Liu et al. J Am Coll Surg. 2016; 222:1009-17). Mice lacking the IAP gene characteristically display an impaired gut barrier and increased intrahepatic inflammatory markers, and these findings become more prevalent with age (Kuhn et al., JCI insight. 2020 5). It has been shown in a mouse alcohol model that mice lacking IAP display accelerated liver steatosis, whereas oral supplementation with IAP alleviates the development of steatosis (Hamarneh et al., Dig Dis Sci. 2017). Given these observations, it was examined whether IAP may rescue the liver from the development of fibrosis in other models of hepatic injury, specifically common bile duct ligation and CCl4 toxicity.

There remains an unmet clinical need for effective and well-tolerated therapies directed to the treatment or prevention of liver fibrosis.

### SUMMARY

Accordingly, in some aspects, the present disclosure provides intestinal alkaline phosphatase (IAP) compositions, including variants thereof, for the treatment, inhibition, and/or prevention of liver fibrosis.

In some aspects, the present disclosure provides methods and compositions for treating, inhibiting, or preventing liver fibrosis secondary to a liver disease or disorder characterized by hepatotoxicity via administering an alkaline phosphatase (AP)-based agent to a subject. In some aspects, the liver disease or disorder characterized by hepatotoxicity is selected from nonalcoholic fatty liver disease (NAFLD), nonalcoholic steatohepatitis (NASH), and acute-on-chronic liver failure (ACLF).

In other aspects, the present disclosure provides methods and compositions for treating or inhibiting liver fibrosis via administering an AP-based agent to a subject. In some embodiments, the liver fibrosis has a non-biliary etiology.

In some embodiments, the liver fibrosis is not characterized by cholestasis. In some embodiments, the liver fibrosis is not characterized by substantially increased integrin expression in biliary epithelial cells, as compared to an undiseased state. In some embodiments, the integrin is integrin αvβ6. In some embodiments, the liver fibrosis is pericentral fibrosis. In some embodiments, the liver fibrosis develops over at least several months of ongoing liver injury. In some embodiments, the liver fibrosis is not caused by age-related physiological alteration of, or related to, intestinal homeostasis. In some embodiments, the intestinal homeostasis is measured by a decrease in ZO-1 protein, ZO-2 protein, occludin, or tight junction proteins, or is measured by an increase in HMGB1 (High Mobility Group Box 1).

In some embodiments, the liver fibrosis is modelled by the Carbon Tetrachloride-4 (CCl4) model. In some embodiments, the liver fibrosis is not modelled by the common bile duct ligation (CBDL) model.

In some embodiments, the methods or compositions prevent or mitigate the development of one or more of cirrhosis, end-stage liver disease, and hepatocellular carcinoma.

In some embodiments, the methods or compositions prevent or mitigate the development of periportal fibrosis, bridging fibrosis, or cirrhosis.

In some embodiments, the subject is afflicted by one or more of insulin resistance, pre-diabetes, type 2 diabetes mellitus, and obesity and does not regularly consume alcohol. In some embodiments, the subject is characterized by hepatocellular ballooning.

In some embodiments, the administration of the AP-based agent results in a decrease or lack of increase in expression and/or activity of one or more of tissue inhibitor of metalloproteinases-1 (TIMP-1), collagen-1, and smooth muscle alpha α-2 actin (ACTA-2). In some embodiments, the administration of the AP-based agent results in a decrease or lack of increase in expression and/or activity of Smooth Muscle Alpha-Actin (α-SMA).

In some embodiments, the AP-based agent is intestinal alkaline phosphatase (IAP). In some embodiments, the IAP is bovine IAP (bIAP). In some embodiments, the bIAP comprises an amino sequence having at least about 90%, or about 95%, or about 97%, or about 98%, or about 99% sequence identity to SEQ ID NO: 11 or any one of SEQ ID NO: 1 to SEQ ID NO: 10. In some embodiments, the bIAP comprises an amino sequence having at least about 97% sequence identity to one of SEQ ID NO: 11 or any one of SEQ ID NO: 1 to SEQ ID NO: 10.

In some embodiments, the AP-based agent is administered enterally or parenterally. In some embodiments, the enteral administration is oral administration.

In some embodiments, the subject is a human patient.

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### BRIEF DESCRIPTION OF THE DRAWINGS

FIGS. 1A-IF show that IAP expression is decreased in liver fibrosis in both mouse and human. Specifically, FIG. 1A shows stool IAP activity from liver cirrhosis patients vs. control measured by pNPP assay; FIG. 1B shows stool IAP level of liver cirrhosis patients in different liver Child-Pugh classifications measured by pNPP assay; FIG. 1C shows stool IAP activity in CBDL induced liver fibrosis model measured by pNPP assay at different time-points after the procedure; FIG. 1D shows stool IAP activity in CCl4 induced liver fibrosis model measured by pNPP assay at different time-points after the procedure; FIG. 1E shows mouse duodenal AKP3 (duodenal IAP) and AKP6 (global IAP gene) gene expression detected by qPCR methods in CBDL induced liver fibrosis model; and FIG. 1F shows mouse duodenal AKP3 (duodenal IAP) and AKP6 (global IAP gene) gene expression detected by qPCR methods in CCl4 induced liver fibrosis model; *: p<0.05. In FIG. 1E and FIG. 1F, for each gene, the left bar is sham and the right bar is the model used (i.e. CBDL or CCl4).

FIGS. 2A-2F show that IAP regulates intestinal tight junction protein expression in models of liver fibrosis in mice. Specifically, FIG. 2A shows terminal ileum tight junction protein genes (ZO-1, ZO-2, ZO-3, Occludin) expression in sham or common bile duct ligated mice detected by qPCR in WT and IAP KO (knock out) animals; FIG. 2B shows serum FITC-dextran level 4-hour after gavage at indicated groups; FIG. 2C shows serum LPS concentration measured by LAL assay; FIG. 2D shows terminal ileum level of tight junction proteins (ZO-1, ZO-2, ZO-3, Occludin) expression after 8 weeks of CCl4 induced liver fibrosis determined by qPCR; FIG. 2E shows serum FITC-dextran level 4-hour after gavage at indicated groups measured by LAL assay; and FIG. 2F shows serum LPS concentration at indicated groups measured by LAL assay; *: P<0.05. In FIG. 2A and FIG. 2D, the order of bars, left to right, is WT Sham, WT, IAP KO Sham, and IAP KO.

FIGS. 3A-3I show that lack of IAP leads to a severe liver fibrosis after CBDL and CCL4 treatments. Specifically, in FIGS. 3A-3C, WT and IAP KO mice underwent CBDL for 3 weeks, and liver levels of ACTA-2 (FIG. 3A), TIMP-2 (FIG. 3B) and Collagen-1 (FIG. 3C) genes were determined by qPCR, and compared with WT mice; in FIGS. 3D-3E, 3 weeks of CBDL mice were tested for fibrillar collagen by Sirius red staining (×100) (FIG. 3D), and expression of α-SMA was determined by immunohistochemistry (×200) (FIG. 3E); in FIGS. 3F-3G, WT and IAP KO mice underwent CCl4 treatment for 8 weeks, and liver levels of TIMP-2 (FIG. 3F) and Collagen-1 (FIG. 3G) genes were determined by qPCR; in FIGS. 3H-3I, 8 weeks of CCl4 injected mice were tested for fibrillar collagen by Sirius red staining (×100) (FIG. 3H), and expression of α-SMA was determined by immunohistochemistry (×200) (FIG. 3I); *: P<0.05.

FIGS. 4A-4F show that IAP supplementation rescues the gut barrier in murine models of liver fibrosis. Specifically, FIG. 4A shows the level of ileal tight junction protein (ZO-1, ZO-2, ZO-3, Occludin) expression in mice treated with vehicle or IAP determined by qPCR, 3 weeks after CBDL; FIG. 4B shows systemic serum FITC-dextran level 4-hour after oral gavage; FIG. 4C shows portal serum LPS concentration measured by LAL assay, 3 weeks after CBDL; FIG. 4D shows the level of tight junction protein (ZO-1, ZO-2, ZO-3, Occludin) expression as determined by qPCR, after 8 weeks of CCl4 induced liver fibrosis; FIG. 4E shows systemic serum FITC dextran after 8 weeks of CCl4 induced liver fibrosis; and FIG. 4F shows portal serum LPS concentration after 8 weeks of CCl4 induced liver fibrosis; *: P<0.05. In FIG. 4A and FIG. 4D, the order of bars, left to right, is WT Sham, WT, and WT+IAP.

FIGS. 5A-5I show that supplementation of IAP prevents the development of liver fibrosis in murine liver fibrosis models. Specifically, FIGS. 5A-5C show liver levels of ACTA-2 (FIG. 5A), TIMP-2 (FIG. 5B) and Collagen-1 (FIG. 5C) genes in WT mice treated with oral vehicle or IAP at 3 weeks after CBDL, determined by qPCR; FIGS. 5D-5E show liver fibrosis tested for fibrillar collagen by Sirius red staining (×100) (FIG. 5D) and expression of α-SMA determined by immunohistochemistry (×200) (FIG. 5E) in mice that underwent CBDL; in FIGS. 5F-5G, WT mice with supplementation of IAP underwent CCl4 treatment for 8 weeks, and levels of TIMP-2 (FIG. 5F) and Collagen-1 (FIG. 5G) gene expression in the liver were determined by qPCR; and in FIGS. 5H-5I, mice treated with 8 weeks of CCl4 were tested for fibrillar collagen by Sirius red staining (×100) (FIG. 5H), and expression of α-SMA was determined by immunohistochemistry (×200) (FIG. 5I); *: P<0.05.

FIGS. 6A-6I show that supplementation of IAP prevents the IAP KO mice development of liver fibrosis. Specifically, in FIGS. 6A-6C, IAP KO mice with oral supplement IAP and 3 weeks after CBDL, liver levels of ACTA-2 (FIG. 6A), TIMP-2 (FIG. 6B) and Collagen-1 (FIG. 6C) genes were determined by qPCR; in FIGS. 6D-6E, mice 3 weeks after CBDL were tested for fibrillar collagen by Sirius red staining (×100) (FIG. 6D) and expression of α-SMA was determined by immunohistochemistry (×200) (FIG. 6E); in FIGS. 6F-6G, IAP KO mice with supplementation of IAP underwent CCl4 treatment for 8 weeks, and levels of TIMP-2 (FIG. 6F) and Collagen-1 (FIG. 6G) gene expression in the liver were determined by qPCR; in FIGS. 6H-6I, mice treated with 8 weeks of CCl4 were tested for fibrillar collagen by Sirius red staining (×100) (FIG. 6H), and expression of α-SMA was determined by immunohistochemistry (×200) (FIG. 6I); *: P<0.05.

FIGS. 7A-7I show that IAP supplementation does not attenuate fibrosis in TLR4-KO mice. Specifically, in FIGS. 7A-7C, 3 weeks after CBDL in TLR4 KO mice, ACTA-2 (FIG. 7A), TIMP-2 (FIG. 7B) and Collagen-1 (FIG. 7C) gene expression in the liver was compared from mice with and without IAP treatment; Sirius red staining (×100) (FIG. 7D) and α-SMA expression (×200) (FIG. 7E) were also compared between the groups; in FIGS. 7F-7I, the CCl4 induced liver fibrosis model was also used in TLR4 mice with IAP in drinking water. After 10 weeks, liver TIMP-2 (FIG. 7F) and Collagen-1 (FIG. 7G) gene expression were detected by qPCR, and liver collagen fiber was detected by Sirius red staining (×100) (FIG. 7H), and α-SMA was detected by immunochemistry method (×200) (FIG. 7I); *: P<0.05.

### DETAILED DESCRIPTION

The present disclosure is directed, in part, to uses of intestinal alkaline phosphatases (IAPs) for the treatment, inhibition, and/or prevention of liver fibrosis.

In some aspects, the present disclosure provides methods and compositions for treating or preventing liver fibrosis secondary to a liver disease or disorder characterized by hepatotoxicity via administering an alkaline phosphatase (AP)-based agent to a subject. In some aspects, the liver disease or disorder characterized by hepatotoxicity is selected from nonalcoholic fatty liver disease (NAFLD), nonalcoholic steatohepatitis (NASH), and acute-on-chronic liver failure (ACLF).

In other aspects, the present disclosure provides methods and compositions for treating or inhibiting liver fibrosis via administering an AP-based agent to a subject. In some embodiments, the liver fibrosis has a non-biliary etiology.

### Liver Fibrosis and Methods of Treatment or Prevention

Without wishing to be bound by theory, liver fibrosis arises from the excessive accumulation of extracellular matrix proteins including collagen that occurs in most types of chronic liver diseases. Non-alcoholic fatty liver disease (NAFLD) is the most common liver disorder in the Western world. It encompasses a disease spectrum of progressive liver disease ranging from non-alcoholic fatty liver (NAFL) to non-alcoholic steatohepatitis (NASH), which can develop with liver fibrosis and cirrhosis. The disease is associated with obesity, and approximately 1 in 3 people in the US have some degree of NAFLD, and on average about 1 in 5 people worldwide have some degree of NAFLD. NAFLD is underdiagnosed since patients are usually asymptomatic even prior to the development of end-stage liver disease (ESLD).

In some embodiments, liver fibrosis subjects can be identified, diagnosed, and/or confirmed, for example, by liver biopsy, for example using the Brunt classification method (Brunt et al., 2007, Modern Pathol., 20: S40-S48) or another classification method described herein or known in the art, ultrasound, magnetic resonance imaging (MRI), transient elastography (FibroScan), quantitation of plasma levels of free eicosanoids, detection and quantitation of one or more biomarkers in a biological sample taken from an individual, including, but not limited to, apolipoprotein F, lipopolysaccharide-binding protein, ficolin-2, apolipoprotein D, kininogen-1, apolipoprotein M, thrombospondin-1, IgG F C-binding protein, cystatin-C, alpha-1-acid glycoprotein 2, leucine-rich alpha-2-glycoprotein, ACY, SHBG, CTSZ, MET, GSN, LGALS3BP, PLAT, CHL1, SERPINC1, SIGLEC7, and SIGLEC14, and those described in US20190339288 and US20170227550, incorporated by reference herein in their entireties. Biomarkers can be detected and quantitated using any suitable methods known to those skilled in the art in view of the present disclosure. Examples of methods for detecting and quantitating biomarkers include, but are not limited to, those described in US2019/0339288 and US2017/0227550, incorporated herein by reference in their entireties. In some embodiments, the sample is a blood sample, serum sample, or plasma sample, although it may include cells, cell lysates, urine, amniotic fluid and other biological fluids.

In some embodiments, subjects are selected for treatment with a composition comprising or consisting of an AP-based agent, as described herein, using a staging method including, but not limited to, the histology activity index (HAI), Knodell, Scheuer, Batts-Ludwig, METAVIR, Brunt, Kleiner, Ishak, or the nonalcoholic fatty liver disease activity score (NAS) scoring systems. In the vast majority of clinico-pathological studies, liver biopsies with fibrosis score ≥2/4 are considered to have clinically significant fibrosis; cirrhosis corresponds to the highest score and the last stage in all systems. Examples of suitable scoring systems are described in Almpanis et al., Ann Gastroenterol. 2016; 29(4):445-453, incorporated herein by reference in its entirety.

In some embodiments, subjects are selected for treatment with a composition comprising or consisting of an AP-based agent, as described herein, using the HAI system, which is an additive score calculated by summing semi-quantitative scores for four individual features: periportal and/or bridging necrosis, hepatocyte degeneration and/or focal necrosis, portal inflammation, and fibrosis. According to HAI, fibrosis is staged using a 5-tier system, with stage 0 corresponding to absence of fibrosis and stage 4 to cirrhosis. Intermediate stages 1 and 3 correspond to fibrous expansion of portal tracts (score 1) and bridging fibrosis (score 3), respectively.

In some embodiments, subjects are selected for treatment with a composition comprising or consisting of an AP-based agent, as described herein, using the Knodell system (Knodell et al., Hepatology. 1981; 1:431-435), which eliminates score 2 from the HAI system and has three stages of fibrosis (portal, bridging, and cirrhosis).

In some embodiments, subjects are selected for treatment with a composition comprising or consisting of an AP-based agent, as described herein, using one of the 5-tier histological staining systems currently in use based on the Knodell fibrosis score, in which fibrosis is scored from 0-4, such as the Scheuer system (no fibrosis, enlarged portal tracts, periportal fibrosis+/−periportal septa, architectural distortion with no obvious cirrhosis, and probable or definite cirrhosis) (Scheuer, J Hepatol. 1991; 13:372-374), the Batts-Ludwig system (no fibrosis, portal/periportal fibrosis, septal fibrosis, bridging fibrosis with architectural distortion, and cirrhosis) (Batts and Ludwig, Am J Surg Pathol. 1995; 19:1409-1417), the METAVIR system (no fibrosis, portal fibrosis without septa, few septa, numerous septa without cirrhosis, and cirrhosis) (Bedossa and Poynard, Hepatology. 1996; 24:289-293), the Brunt system (no fibrosis, zone 3 (perisinusoidal, focal or extensive), zone 3 and focal/extensive portal-based fibrosis, same as previous two, but with bridging fibrosis, and cirrhosis) (Brunt et al., Am J Gastroenterol. 1999; 94:2467-2474), or the Kleiner system (Kleiner et al., Hepatology. 2005; 41:1313-1321).

In some embodiments, subjects are selected for treatment with a composition comprising or consisting of an AP-based agent, as described herein, using a 7-tier histological staining systems currently in use based on the Knodell fibrosis score, in which fibrosis is scored from 0-6, such as the Ishak system (no fibrosis, expansion of some portal areas with or without septa, expansion of most portal areas with or without septa, expansion of most portal areas with portal-portal bridging, expansion of most portal areas with portal-portal and portal-central bridging, bridging with occasional nodules, and cirrhosis) (Ishak et al., J Hepatol. 1995; 22:696-699).

In some embodiments, subjects are selected for treatment with a composition comprising or consisting of an AP-based agent, as described herein, using the Laennec system (Kim et al., J Hepatol. 2012 Sep; 57(3):556-63), which is a modification of the METAVIR system that subdivides stage 4 (cirrhosis) into three sub-stages (4A mild or probable cirrhosis, 4B moderate cirrhosis, and 4C severe cirrhosis), taking into consideration the width of the fibrous septa and the size of cirrhotic nodules).

In various embodiments, subjects are selected for treatment with a composition comprising or consisting of an AP-based agent, as described herein, via the NAFLD Activity Score (NAS), which differentiates between NASH and NAFLD. NAS is the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and >5 corresponds to NASH. The biopsy is also scored for fibrosis (0 to 4).

In some embodiments, the subject has liver fibrosis, i.e., has been diagnosed as having liver fibrosis or is suspected as having liver fibrosis, e.g., shows symptoms of liver fibrosis.

“Liver fibrosis” can be used interchangeably with “hepatic fibrosis.” Liver fibrosis is a wound healing response characterized by the excessive accumulation of scar tissue (i.e. extracellular matrix) in the liver. Liver fibrosis may or may not be seen in NASH and is where normal structural elements of tissues are replaced with excessive amounts of non-functional scar tissue. Hepatic fibrosis can be caused by various factors including fatty liver, alcohol and viruses. Liver fibrosis is usually staged by severity using various staging methods such as the HAI, Knodell, Scheuer, Batts-Ludwig, METAVIR, Brunt, Kleiner, Ishak, or NAS scoring systems.

In some embodiments, the subject has liver fibrosis secondary to a liver disease or disorder characterized by hepatotoxicity. In some embodiments, the liver disease or disorder characterized by hepatotoxicity is selected from NAFLD, NAFL, NASH, and ACLF. In some embodiments, the subject has liver fibrosis characterized by a non-biliary etiology. In some embodiments, the subject has pericentral liver fibrosis.

In some embodiments, the subject has NAFLD, i.e., has been diagnosed as having NAFLD or is suspected as having NAFLD, e.g., shows symptoms of NAFLD. In some embodiments, “nonalcoholic fatty liver disease” or “NAFLD” refers to a condition in which fat is deposited in the liver (hepatic steatosis), with or without inflammation and fibrosis, in the absence of excessive alcohol use. In some embodiments, NAFLD is suspected during a routine checkup, monitoring of metabolic syndrome and obesity, or monitoring for possible side effects of drugs (e.g., cholesterol lowering agents or steroids). In some instances, liver enzymes such AST and ALT are high. In some embodiments, a subject is diagnosed following abdominal or thoracic imaging, liver ultrasound, or magnetic resonance imaging. In some embodiments, other conditions such as excess alcohol consumption, hepatitis C, and Wilson's disease have been ruled out prior to an NAFLD diagnosis. In some embodiments, a subject has been diagnosed following a liver biopsy. In some embodiments, a subject has been diagnosed using a staging method including, but not limited to, the HAI, Knodell, Scheuer, Batts-Ludwig, METAVIR, Brunt, Kleiner, Ishak or NAS scoring systems. Other suitable methods of diagnosing NAFLD include those described in Other suitable methods for diagnosing liver fibrosis are described in US20180031585, US20190339288, and US20170227550, incorporated by reference herein in their entireties.

In some embodiments, the subject has NAFL, i.e., has been diagnosed as having NAFL or is suspected as having NAFL, e.g., shows symptoms of NAFL. “Non-alcoholic fatty liver” or “NAFL” is also referred to as simple steatosis. NAFL is diagnosed when a patient has an accumulation of fat (triglycerides) and other lipids in the hepatocytes of the liver. Disordered fatty acid metabolism leads to steatosis and can be caused by various factors such as insulin resistance in diabetes, lack of exercise and excess food intake where there is an imbalance between calorific intake and combustion. In some embodiments, NAFL is diagnosed by a method described above for NAFLD in general.

In some embodiments, the subject has steatosis, i.e., has been diagnosed as having steatosis or is suspected as having steatosis, e.g., shows symptoms of steatosis. In some embodiments, “steatosis” and “non-alcoholic steatosis” are used interchangeably, and include mild, moderate, and severe steatosis, without inflammation or fibrosis, in the absence of excessive alcohol use. In some embodiments, the steatosis is mild, moderate, or severe steatosis. Table 1 shows exemplary classification of mild, moderate, and severe steatosis. In some embodiments, steatosis is diagnosed by a method described above for NAFLD in general.

In some embodiments, the subject has NASH, i.e., has been diagnosed as having NASH or is suspected as having NASH, e.g., shows symptoms of NASH. “Non-alcoholic steatohepatitis” or “NASH” is a more aggressive stage of NAFLD where there is hepatic inflammation accompanied with steatosis. Exemplary methods of determining the stage of NASH are described, for example, in Kleiner et al., 2005, Hepatology, 41(6):1313-1321, and Brunt et al., 2007, Modern Pathol., 20: S40-S48. In some embodiments, the NASH is stage 1, 2, 3, or 4 NASH. Table 1 shows exemplary classification of stage 1, 2, 3, and 4 NASH. In some embodiments, NASH is diagnosed by a method described above for NAFLD in general. In some embodiments, advanced fibrosis is diagnosed in a subject with NAFLD, for example, according to Gambino R, et. al. Annals of Medicine 2011; 43(8):617-49.

TABLE 1
Subclassification of Steatosis and NASH Stage Groups

| | Steatosis | Inflammation | Ballooning | Fibrosis |
|---|---|---|---|---|
| Mild steatosis | 1 | 0 or 1 | 0 | 0 |
| Moderate | 2 | 0, 1 or 2 | 0 | 0 |
| steatosis | | | | |
| Severe steatosis | 3 | 0 or 2 | 0 | 0 |
| NASH stage 1 | 1, 2 or 3 | 0 or 1 | 1 | 1 |
| NASH stage 2 | 1, 2 or 3 | 0, 1 or 2 | 2 | 2 |
| NASH stage 3 | 1, 2 or 3 | 0, 1 or 2 | 2 | 3 |
| (bridging) | | | | |
| NASH stage 4 | 1, 2 or 3 | 0, 1 or 2 | 2 | 4 |
| (cirrhosis) | | | | |

In some embodiments, the subject has ACLF, i.e., has been diagnosed as having ACLF or is suspected as having NASH, e.g., shows symptoms of ACLF. “Acute-on-chronic liver failure” or “acute chronic liver failure” or “ACLF” is a distinct clinical entity encompassing an acute deterioration of liver function in subjects with cirrhosis, often decompensated cirrhosis, which is usually associated with a precipitating event and results in the failure of one or more organs and high short term mortality. Unregulated inflammation is thought to be a major contributing factor. A characteristic feature of ACLF is its rapid progression, the requirement for multiple organ supports and a high incidence of short and medium term mortality of 50-90%. ACLF is diagnosed by use of the Chronic Liver Failure (CLiF) Consortium criteria, NACSELD criteria or APASL criteria. Previously validated scores to assess disease severity include Child-Pugh (CP) classification, Model for End Stage Liver Disease (MELD) and the CLiF Consortium Acute Decompensation (CLIF-C AD) score.

In some embodiments, the liver fibrosis is not characterized by cholestasis. In some embodiments, “cholestasis” means the disease or symptoms comprising impairment of bile formation and/or bile flow. In some embodiments, “cholestatic liver disease” means a liver disease associated with cholestasis. Cholestatic liver diseases are often associated with jaundice, fatigue, and pruritis. Biomarkers of cholestatic liver disease include elevated serum bile acid concentrations, elevated serum AP, elevated gamma-glutamyltranspeptidease, elevated conjugated hyperbilirubinemia, and elevated serum cholesterol.

In some embodiments, the liver fibrosis is not characterized by substantially increased integrin expression in biliary epithelial cells, as compared to an undiseased state. Integrins are receptors mediating interactions between the cell and their surrounding ECM, and comprise a family of transmembrane cellular protein receptors composed of noncovalently linked α- and β-subunits that can form at least 24 different combinations that are differentially expressed by various cell types and recognize multiple ligands. In some embodiments, the integrin is integrin α1β1, integrin α2β1, integrin α3β1, integrin α4β1, integrin α5β1, integrin α6β1, integrin α7β1, integrin α8β1, integrin α9β1, integrin αvβ1, integrin α1β2, integrin αLβ2, integrin αMβ2, integrin αXβ2, integrin αvβ3, integrin αIIbβ3, integrin α6β4, integrin αvβ5, integrin αvβ6, integrin α4β7, integrin αEβ7, or integrin αvβ8. In some embodiments, the integrin is integrin αvβ6 or integrin αvβ8. In some embodiments, an “undiseased state” refers to the state of an individual in which the disease or condition of interest (such as liver fibrosis, NAFLD, NAFL, NASH, ACLF, etc.) is not detectable by conventional diagnostic methods.

In some embodiments, the liver fibrosis develops over at least several months of ongoing liver injury. In some embodiments, the fibrosis develops over at least 2, 3, 4, 5, 6, 7, 8, 19, 10, 11, 12, 13, 14, 15, 16, 17, 18, 19, 20, 21, 22, 23, or 24 months of ongoing liver injury.

In some embodiments, the liver fibrosis develops over at least 2, 2.5, 3, 3.5, 4, 4.5, 5, 5.5, 6, 6.5, 7, 7.5, 8, 8.5, 9, 9.5, 10 years, or more, of ongoing liver injury.

In some embodiments, the liver fibrosis is not caused by age-related physiological alteration of, or related to, intestinal homeostasis. In some embodiments, “intestinal homeostasis” means the maintenance of the equilibrium between a subject and its intestinal microbiota. Intestinal homeostasis depends upon the mucosal barrier and permeability of the mucosal barrier. Lack of intestinal homeostasis is sometimes known as intestinal dysbiosis. In some embodiments, the intestinal homeostasis is measured by a decrease in one or more tight junction-associated proteins, including, but not limited to, ZO-1 protein, ZO-2 protein, ZO-3 protein, occludin, 7H6, AF-6, or tight junction proteins. In some embodiments, intestinal homeostasis is measured by an increase in HMGB1 (High Mobility Group Box 1) protein.

In some embodiments, the liver fibrosis is secondary, or caused by alcoholism.

In some embodiments, the liver fibrosis is not modelled by the common bile duct ligation (CBDL) model.

In some embodiments, the liver fibrosis is modelled by the Carbon Tetrachloride-4 (CCl4) model.

In some embodiments, administration of a composition comprising or consisting of an AP-based agent results in a decrease or lack of increase in expression or activity of one or more of tissue inhibitor of metalloproteinases-1 (TIMP-1), collagen-1, and smooth muscle alpha α-2 actin (ACTA-2). In some embodiments, administration of a composition comprising an AP-based agent results in a decrease or lack of increase in expression or activity of smooth muscle alpha-actin (α-SMA) protein. Any method known in the art can be used to measure the levels of the genes or proteins of interest in a biological sample. In some embodiments, levels of the genes of interest are measured by assessing the level of mRNA in a biological sample using any method known in the art, including, but not limited to, quantitative PCR. In some embodiments, levels of the proteins of interest are measured by assessing the level of protein in a biological sample using any method known in the art, including, but not limited to, an immunoassay (e.g., enzyme linked immunosorbent assay (ELISA), a radioimmunoassay (RIA), Western blotting, or dot blotting), or mass spectrometry. Suitable methods for measuring the activity of TIMP-1, collagen-1, and α-SMA are known in the art.

In some embodiments, administration of a composition comprising or consisting of an AP-based agent prevents or mitigates the development of complications of liver fibrosis. For example, in some embodiments, administration of a composition comprising or consisting of an AP-based agent prevents or mitigates the development of cirrhosis, end-stage liver disease, periportal fibrosis, or bridging fibrosis. “Hepatic cirrhosis” or “cirrhosis” is the most severe form of liver scarring and, unlike hepatic fibrosis, is nodular and causes irreversible architectural damage to the liver. Cirrhosis is a major risk factor for hepatocellular carcinoma (HCC) and, at this stage of liver cancer, the only curative approach is liver transplantation. In embodiments, “end-stage liver disease” and “chronic liver failure” refer to liver failure that usually develops over a period of years and is caused by a repeated insult to the liver, which slowly damages the organ. According to the METAVIR staging system, stage 3 fibrosis (F3) is defined as “bridging fibrosis” evidenced by fibrotic bridging that extends across lobules, between portal areas, and between portal areas and central veins. It is an extension from stage 2 fibrosis or “periportal fibrosis” in which fibrosis is limited to periportal or perivenular areas. The hepatic architecture remains relatively intact. When fibrosis progresses to and distorts the liver architecture with formation of nodules, it is considered stage 4 fibrosis (F4) or cirrhosis.

In some embodiments, administration of a composition comprising or consisting of an AP-based agent prevents, inhibits, and/or mitigates the development of a cancer associated with liver fibrosis. In some embodiments, administration of a composition comprising an AP-based agent prevents or mitigates the development of hepatocellular carcinoma. The terms “hepatocellular carcinoma,” “HCC,” and “hepatoma” are used interchangeably herein to refer to cancer that arises from hepatocytes.

In some embodiments, administration of a composition comprising or consisting of an AP-based agent treats, prevents, inhibits, and/or mitigates one or more of liver fibrosis, cirrhosis, end-stage liver disease, hepatocellular carcinoma, periportal fibrosis, and bridging fibrosis.

In embodiments, “treatment” or “mitigation” in the context of the present disclosure refers to at least one of the following: prevent, reduce, or eliminate the disease or condition of interest (such as liver fibrosis, NAFLD, NAFL, NASH, ACLF, etc.); prevent, reduce, or eliminate one or more symptoms or side effects associated with the disease or condition of interest; decrease the severity of at least one undesired symptom or side effect associated with the disease or condition of interest; delay disease progression, including delay in the progression from one disease stage to another of the disease or condition of interest; improve liver function in a subject with a disease or condition of interest, as measured by a hepatic function panel test; improve prognosis of a subject with a disease or condition of interest, for example as measured by a Model for End-Stage Liver Disease (MELD) score, or by one or more of the HAI, Knodell, Scheuer, Batts-Ludwig, METAVIR, Brunt, Kleiner, NAS, and Ishak systems; slow the rise in MELD, HAI, Knodell, Scheuer, Batts-Ludwig, METAVIR, Brunt, Kleiner, NAS, and Ishak scores in a subject with a disease or condition of interest; prevent or slow the deterioration of any damage caused to the liver tissue, such as the accumulation of fibrotic scar tissue, by factors known to cause cirrhosis.

In embodiments, “treatment” is also meant to refer to preventive, inhibitory, and/or prophylactic treatment, meaning that a person known to have a disease or condition of interest (such as liver fibrosis, NAFLD, NAFL, NASH, ACLF, etc.), or to be at risk for developing a disease or condition of interest, is administered with a composition comprising or consisting of an AP-based agent, even before manifestation of the disease or condition of interest to prevent or inhibit its occurrence.

In some embodiments, administration of a composition comprising or consisting of an AP-based agent improves the symptoms and/or sense of well-being of the subject. In some embodiments, administration of a composition comprising an AP-based agent prevents, reduces, or eliminates a symptom associated with liver fibrosis including, but not limited to, cystic changes in the liver (characterized, for example, by dilatation of the bile ducts); abdominal distension; swelling of the legs; fullness; back pain; clinical signs of hepatic fibrosis; cyst infection, hemorrhage, and rupture; portal hypertension; easy bruising and bleeding; itching; poor appetite, nausea and weight loss; fatigue, confusion, and weakness; and jaundice.

In some embodiments, administration of a composition comprising or consisting of an AP-based agent reduces the likelihood of a shortened lifespan associated with liver fibrosis.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon.

In some embodiments, methods of the disclosure are useful in treatment of a human patient. In some embodiments, the human is a pediatric human. In other embodiments, the human is an adult human. In other embodiments, the human is a geriatric human. In some embodiments, the human is a female. In some embodiments, the human is a male.

In some embodiments, the patient is at risk of developing NAFLD, including, but not limited to, a subject with one or more NAFLD comorbidities, such as obesity, abdominal obesity, metabolic syndrome, cardiovascular disease, and diabetes. In some embodiments, the patient is afflicted by one or more of insulin resistance, pre-diabetes, type 2 diabetes mellitus, and obesity. In some embodiments, the subject does not regularly consume alcohol. In some embodiments, the patient is characterized by hepatocellular ballooning. In some embodiments, “hepatocellular ballooning” refers to the process of hepatocyte cell death.

In some embodiments, the human patient has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, the form of the AP-based agent, including pharmaceutical compositions comprising or consisting of the AP-based agent, the route of administration, the dosage and the regimen, including administration frequency and duration, depend upon the condition to be treated, the severity of the illness, the age, weight, and sex of the patient, etc. Alkaline phosphatases of the instant disclosure may be formulated for enteral, oral, intranasal, parenteral, intravenous, intramuscular or subcutaneous administration and the like. In some embodiments, the AP-based agent is administered enterally or parenterally. In some embodiments, the enteral administration is oral administration.

### Alkaline Phosphatases (APs), AP-Based Agents, and Compositions

The present disclosure is directed, at least in part, to pharmaceutical compositions, formulations, and uses of one or more alkaline phosphatases. Alkaline phosphatases are dimeric metalloenzymes that catalyze the hydrolysis of phosphate esters and dephosphorylate a variety of target substrates at physiological and higher pHs. Illustrative APs that may be utilized in the present disclosure include, but are not limited to, intestinal alkaline phosphatase (IAP; e.g., calf IAP or bovine IAP, chicken IAP, and goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In various embodiments, the present disclosure contemplates the use of mammalian alkaline phosphatases including, but are not limited to, intestinal alkaline phosphatase (IAP), placental alkaline phosphatase (PLAP), germ cell alkaline phosphatase (GCAP), and the tissue non-specific alkaline phosphatase (TNAP).

### Intestinal Alkaline Phosphatase (IAP)

In some embodiments, the alkaline phosphatase is IAP. IAP is produced in the proximal small intestine and is bound to the enterocytes via a glycosyl phosphatidylinositol (GPI) anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In an embodiment, the IAP is human IAP (hIAP). In an embodiment, the IAP is calf IAP (cIAP), also known as bovine IAP (bIAP). There are multiple isozymes of bIAP, for example, with bIAP II and IV having higher specific activity than bIAP I. In an embodiment, the IAP is any one of the cIAP or bIAP isozymes (e.g., bIAP I, II, and IV). In an embodiment, the IAP is bIAP II. In another embodiment, the IAP is bIAP IV.

In various embodiments, the IAP of the present disclosure has greater specific enzymatic activity than commercially-available APs, e.g., calf IAP (cIAP).

Also included within the definition of IAPs are IAP variants. An IAP variant has at least one or more amino acid modifications, generally amino acid substitutions, as compared to the parental wild-type sequence. In various embodiments, the IAP of the disclosure may comprise an amino acid sequence having one or more amino acid mutations relative to any of the protein sequences described herein. In some embodiments, the one or more amino acid mutations may be independently selected from substitutions, insertions, deletions, and truncations. In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured by any suitable assay known in the art.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general).

Mutations can be made to the IAP of the disclosure to select for agents with desired characteristics. For examples, mutations may be made to generate IAPs with enhanced catalytic activity or protein stability. In various embodiments, directed evolution may be utilized to generate IAPs of the disclosure. For example, error-prone PCR and DNA shuffling may be used to identify mutations in the bacterial alkaline phosphatases that confer enhanced activity.

In some embodiments, an IAP of the invention comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 1.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 2.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 3.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 4.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 5.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 6.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 7.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 8.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 9.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 10.

In some embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 11.

In various embodiments, the IAP is human IAP (hIAP). In some embodiments, the IAP is hIAP comprising the amino acid sequence of SEQ ID NO: 1 or a variant thereof, as long as the hIAP variant retains at least 75, 80, 85, 90, 95, 96, 97, 98, 99 or 100% of the phosphatase activity as compared to the wild type enzyme using any suitable assay known in the art.

In some embodiments, the IAP is bovine IAP (bIAP). In various embodiments, the IAP is bovine IAP II (bIAP II) or a variant as described herein, as long as the bIAP variant retains at least 75, 80, 85, 90, 95, 96, 97, 98, 99, or 100% of the phosphatase activity using any suitable assay known in the art. In an embodiment, the bIAP II comprises the signal peptide and carboxy terminus of bIAP I. In an embodiment, the bIAP II comprises an aspartate at position 248 (similar to bIAP IV). In an embodiment, the bIAP II comprises the amino acid sequence of SEQ ID NO: 2 or a variant thereof, as long as the bIAP II variant retains at least 75, 80, 85, 90, 95, 96, 97, 98, 99 or 100% of the phosphatase activity as compared to the wild type enzyme using any suitable assay known in the art. In various embodiments, the bIAP II comprises the amino acid sequence of SEQ ID NO: 11 or a variant thereof, as long as the bIAP II variant retains at least 75, 80, 85, 90, 95, 96, 97, 98, 99 or 100% of the phosphatase activity as compared to the wild type enzyme using any suitable assay known in the art.

### Fusion Proteins

In various embodiments, the present disclosure provides chimeric proteins. In some embodiments, the present disclosure provides chimeric fusion proteins. For example, in various embodiments, the present disclosure provides an isolated or recombinant alkaline phosphatase comprising a crown domain and a catalytic domain, wherein said crown domain and said catalytic domain are obtained from different alkaline phosphatases (e.g., human and bovine alkaline phosphatases). In other embodiments, the alkaline phosphatases are both human APs. In certain embodiments, the present disclosure provides for recombinant fusion proteins comprising human IAP and a domains of human placental alkaline phosphatases. In certain embodiments, the present disclosure provides for chimeric hIAP-placenta fusion proteins.

In various embodiments, the AP-based agent of the disclosure is a fusion protein. In some embodiments, the AP-based agent comprises an alkaline phosphatase fused to a protein domain that replaces the GPI anchor sequence. In some embodiments, the alkaline phosphatase is fused to a protein domain that promotes protein folding and/or protein purification and/or protein dimerization and/or protein stability. In various embodiments, the AP-based agent fusion protein has an extended serum half-life.

In an embodiment, the alkaline phosphatase is fused to an immunoglobulin Fc domain and/or hinge region. In various embodiments, the immunoglobulin Fc domain and/or hinge region is derived from the Fc domain and/or hinge region of an antibody (e.g., of IgG, IgA, IgD, and IgE, inclusive of subclasses (e.g. IgG1, IgG2, IgG3, and IgG4, and IgAQ1 and IgA2)). In an embodiment, the AP-based agent of the disclosure comprises an alkaline phosphatase fused to the hinge region and/or Fc domain of IgG.

In various embodiments, the AP-based agent of the disclosure is a pro-enzyme. In an embodiment, the activity of the proenzyme is suppressed by a carboxy terminus. In an embodiment, protease removal of the carboxy terminus reactivates the enzymatic activity of the alkaline phosphatase. In an embodiment, the pro-enzyme is more efficiently secreted than the enzyme without the carboxy terminus.

In some embodiments, for generation of the pro-enzyme, the native carboxy terminus of the alkaline phosphatase is replaced with the analogous sequence from hPLAP. In some embodiments, a mutation is made in the hydrophobic carboxy tail to promote protein secretion without cleavage of the carboxy terminus. In an illustrative embodiment, a single point mutation such as a substitution of leucine with e.g., arginine is generated in the hydrophobic carboxy terminus to result in secretion of the enzyme without removal of the carboxy terminus.

### Methods of Making APs

The APs of the disclosure are made using standard molecular biology techniques. For example, nucleic acid compositions encoding the APs of the disclosure are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

As is known in the art, the nucleic acids encoding the components of the disclosure can be incorporated into expression vectors as is known in the art, and depending on the host cells, used to produce the AP compositions of the disclosure. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the disclosure are then transformed into any number of different types of host cells as is well known in the art, including mammalian, bacterial, yeast, insect and/or fungal cells, with mammalian cells (e.g. CHO cells), finding use in many embodiments.

The APs of the disclosure are made by culturing host cells comprising the expression vector(s) as is well known in the art. Once produced, traditional purification steps are performed.

### Kits

The disclosure provides kits that can simplify the administration of any agent described herein. An illustrative kit of the disclosure comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the disclosure and an effective amount of another composition, such those described herein.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present disclosure, or embodiments thereof, can alternatively be described using alternative terms such as “consisting of” or “consisting essentially of” As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect can be determined empirically in accordance with conventional procedures for the particular purpose. Generally, for administering therapeutic agents (e.g., microbiome-modulating agents and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays or measurements or methane production in stool samples. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: A Role for Intestinal Alkaline Phosphatase in Preventing Liver Fibrosis

Liver fibrosis is frequently associated with gut barrier dysfunction, and the lipopolysaccharides (LPS)-TLR4 pathway is common to the development of both. Intestinal alkaline phosphatase (IAP) has the ability to detoxify LPS, as well as maintain intestinal tight junction proteins and gut barrier integrity. Therefore, the ability of IAP to prevent liver fibrosis was examined.

### Methods

Stool IAP activity from cirrhotic patients was determined. Common bile duct ligation (CBDL) and Carbon Tetrachloride-4 (CCl4)-induced liver fibrosis models were used in WT, IAP knockout (KO), and TLR4 KO mice supplemented with or without exogenous IAP in their drinking water. The gut barrier function and liver fibrosis markers were tested.

CCl4 is the most widely used hepatotoxin in the study of liver fibrosis and cirrhosis in rodents. In many aspects, it mimics human chronic disease associated with toxic damage. Hepatic biotransformation of CCl4 relies on CYP2E1 and yields the trichloromethyl radical, which is involved in several free radical reactions and lipid peroxidation processes that contribute to an acute phase reaction characterized by necrosis of centrilobular hepatocytes, the activation of Kupffer cells and the induction of an inflammatory response. This sequence is associated with the production of several cytokines, which promote activation of HSCs and hence liver fibrosis (Yanguas et al., Arch Toxicol. 2016; 90(5):1025-1048).

CBDL causes cholestatic injury and periportal biliary fibrosis. CBDL consists of a doubly ligated bile duct transected between two ligatures. The obstruction of the bile duct evokes increases in biliary pressure, mild inflammation, and cytokine secretion by biliary epithelial cells, thus generating cholestasis. This results in proliferation of biliary epithelial cells, an increase of expression of fibrogenic markers, including TIMP-1, α-SMA, collagen 1 and TGFβ1, and accumulation of B-cells and T-cells in the portal tracts, generating reactive oxygen species (ROS) and liver damage (Yanguas et al., Arch Toxicol. 2016; 90(5):1025-1048).

### Human Samples Collection

Stool samples were collected from patients with liver cirrhosis hospitalized in the Liver Disease Center at the Second Affiliated Hospital of Xi'an Jiaotong University. Control stool samples were collected from patients diagnosed with inguinal hernia, gallstones, and thyroid nodular goiter and with no cirrhosis seen in the surgical clinic at Department of General Surgery, Second Affiliated Hospital of the Xi'an Jiaotong University before their operation.

### Animals

IAP KO mice (C57BL/6 background) mice were generated by the Sanford-Burnham Medical Research Institute (La Jolla, CA) (Narisawa et al., Mol Cell Biol. 2003; 23:7525-30) and TLR4 KO (C57BL/6 background) mice were generously gifted from the Mucosal Immunology and Biology Research Center at Massachusetts General Hospital. IAP KO and TLR4 KO littermates were bred at the specific-pathogen-free (SPF) rooms within the Center for Comparative Medicine (CCM) at the Massachusetts General Hospital (MGH) with 12-hour light-dark cycles. All animal experiments (protocol 2017N000152) were reviewed and approved by the Institutional Animal Care and Use Committee at MGH.

### Oral IAP in Drinking Water

IAP was dissolved in autoclaved tap water to a final concentration of 200 IU/mL. IAP was given 4 days prior to bile common duct ligation or CCl4 injection and then continued for the remainder of the experiment. The drinking water supplemented with IAP was changed every day.

### IAP Activity Assay

Stool samples were diluted at a ratio of 1:30 (weight:volume) by stool dilution buffer (10 mM Tris HCl, PH 8.0; 1 mM MgCl2; 10 μM ZnCl2) then homogenized with glass beads for 10 min. The homogenate was then centrifuged at 10,000 g for 10 min and the supernatant was taken for analysis. Total protein concentration was measured by the Bradford assay. To measure IAP activity, 25 μL or supernatant was added to 175 μL pNpp solution (1 mL 1 M Tris-HCl pH 8.0, 1 M MgCl2, 10 mM ZnCl2, 186 mg pNpp, 99 mL Water) and OD405 was measured over time. In separate wells, the selective IAP inhibitor phenylalanine (10 mM) was added to the pNpp solution. Bovine IAP (Sigma) was used to create a standard curve. Change in OD405 over time was normalized to total protein concentration for each sample, and the result with phenylalanine was subtracted from the result without phenylalanine. The final result was reported as units IAP/mg protein.

### Statistical Analysis

Prism 7.0 software (GraphPad, San Diego, CA) was used to analyze the data, shown as mean±SD, and a two-tailed student's t-test or one-way analysis of variance was used to compare groups. A p value<0.05 was considered as significantly different.

### Bile Duct Ligation

Common bile duct ligation was performed as follows and 12 mice in each group. Male mice aged 10-11 weeks were anesthetized using isoflurane inhalation. The abdomen was clipped and prepared in sterile fashion using 70% ethanol. A longitudinal midline laparotomy incision about 2.5 cm was made and the common bile duct was dissected from the portal vein and then doubly ligated with 6-0 vicryl (Ethicon). The abdomen was then closed in two layers using running 5-0 vicryl suture. Sham operated animals underwent an identical procedure with the exception of the common bile duct ligation. Postoperatively, animals were resuscitated with 0.5 mL of normal saline injected subcutaneously and returned to clean cages with water and food ad libitum. Buprenorphine 0.24 mg/kg was injected subcutaneously every 12 hours for the first 48 hours after operation. All the mice were euthanized 3 weeks following the procedure.

### CCl4 Injection

CCl4 (Sigma) was dissolved in olive oil (Sigma) 1:4. Male mice (aged 7-8 weeks) were injected intraperitoneally with 2.5 L/g twice per week (12 mice in each group). The mice were euthanized at 8 weeks, while TLR4 KO mice were euthanized at 10 weeks.

### Serum LPS Concentration Determination

LPS concentration was detected by a commercial chromogenic LPS detection kit (Thermofisher) using the manufacturer's protocol. Briefly, serum samples diluted 1:50 (total volume 50 L) were incubated with 50 μL LAL at 37 degrees for 10 minutes, followed by 100 μL of Chromogenic Substrate solution incubated at 37 degrees for 6 minutes. After adding 100 μL acetic acid, absorbance was measured at 410 nm and LPS concentration was reported as IU/mL.

### Immunochemistry

Fresh tissue was fixed in 4% paraformaldehyde solution overnight, dehydrated by ethanol, and then embedded in paraffin. Slices were cut at room temperature and deparaffinized with xylene substitution, and re-hydrated in ethanol. Antigens were unmasked by 10 mM sodium citrate in a decloaking chamber and incubated at 0.5% H₂O₂ in methanol for 30 min, followed by cooling down to room temperature. Tissue was blocked in normal goat serum (DakoCytomation) diluted 1:3 and incubated with primary antibody overnight at 4 degrees Celsius. Cuts were then incubated with secondary antibody for 1 hour at room temperature and washed with PBS. Slides were developed with ABC (Vector lab) DAB. The reaction was stopped with deionized water and then slides were counterstained with hematoxylin for 6 min. Lastly, slides were dehydrated in ethanol followed by xylene substitute, then mounted.

qPCR

Tissues were homogenized in Trizol solution (Thermo Fisher), and RNA was extracted per the manufacturer's protocol. Extracted RNA was then treated with DNAse with Turbo DNA free kit (Thermofisher) per manufacturer's protocol. 1 g RNA was used to make cDNA with iScript™ Reverse Transcription Supermix (Bio rad). The final qPCR reaction system was comprised of 2 μL cDNA, primers, water and iQ™ SYBR Green Supermix to a final volume of 20 μL. Expression of target gene mRNA was compared with GAPDH, and ΔΔCt method was used to provide relative quantity of gene expression. The average expression of the target gene in the control group was set as 1.

### Intestinal Permeability Evaluated by FITC Dextran

Intestinal permeability was measured using low molecular weight FITC labeled dextran, which permeates across an injured epithelial barrier. 4-7 kD FITC-dextran was dissolved to 75 mg/mL and administered by oral gavage to mice at dose of 300 mg/kg 4 hours prior to sacrifice. Blood was obtained via cardiac puncture after euthanasia, allowed to clot for 30 minutes at room temperature, and then centrifuged to obtain serum. 25 L of serum was then diluted by equal volume of PBS and serum FITC was determined by measuring excitation at 485 nm and emission at 528 nm. Serum FITC is reported as ng/mL of serum.

### Results

Briefly, human stool IAP activity was decreased in the setting of liver cirrhosis. In mice, IAP activity and genes expression decreased after CBDL and CCl4 exposure. Intestinal tight junction related genes and gut barrier function were impaired in both models of liver fibrosis. Oral IAP supplementation attenuated the decrease in small intestine tight junction protein gene expression and gut barrier function. Liver fibrosis markers were significantly higher in IAP KO compared to WT mice in both models, while oral IAP rescued liver fibrosis in both WT and IAP KO mice. In contrast, IAP supplementation did not attenuate fibrosis in TLR4 KO mice in either model.

Fecal IAP Activity Decreased in Humans with Liver Cirrhosis

A total of 18 control patients without cirrhosis and 86 patients with liver cirrhosis had stool samples collected. The mean age of liver cirrhosis patients was 48.08±10.16 with 51 males and 35 females. The etiology of liver diseases included hepatitis B virus (n=53), hepatitis C virus (n=12), HBV and HCV (n=3), alcoholic cirrhosis (n=6), autoimmune cirrhosis (n=5), cholestatic cirrhosis (n=4), and unknown causes (n=3). The Child-Pugh classifications of these patients were as follows: 27 class A patients, 30 class B patients, and 29 class C patients. The non-cirrhosis control samples were collected from patients with biliary colic (n=8), inguinal hernia (n=6) and thyroid nodular goiter (n=4) with a mean age of 45.39±12.52. The liver function was class A in all control group patients. The characteristics of these patients are shown in Table 1.

TABLE 1
Characters for Patients with and without Liver Cirrhosis

| | Cirrhosis | | | | |
|---|---|---|---|---|---|
| | total | A class | B class | C class | No-cirrhosis |
| Number | 86 | 27 | 30 | 29 | 18 |
| Male | 51 | 16 | 16 | 19 | 10 |
| Female | 35 | 11 | 14 | 10 | 8 |
| Hepatitis B | 56 | 15 | 19 | 22 | |
| Hepatitis C | 15 | 5 | 6 | 4 | |
| Alcoholic | 6 | 3 | 2 | 1 | |
| Autoimmune | 5 | 3 | 1 | 1 | |
| Cholestatic | 4 | 1 | 1 | 2 | |
| Unknown | 3 | 1 | 2 | 0 | |
| Smoke | 24 | 11 | 6 | 7 | 4 |
| Duration of | 18.81 ± 25.87 | 14.74 ± 21.04 | 16.55 ± 21.37 | 24.65 ± 32.65 | |
| disease | | | | | |
| (months) | | | | | |
| Total bilirubin | 53.70 ± 54.82 | 23.56 ± 8.17 | 34.23 ± 27.13 | 101.90 ± 67.88 | 14.83 ± 5.35 |
| (μmol/L) | | | | | |
| Scr (μmol/L) | 67.94 ± 34.30 | 63.59 ± 23.26 | 62.07 ± 32.75 | 78.06 ± 42.88 | 55.89 ± 18.81 |
| Platelet | 95.77 ± 58.70 | 114.44 ± 59.40 | 93.13 ± 61.10 | 81.12 ± 54.45 | 210.44 ± 44.03 |
| (×109/L) | | | | | |
| ALT (U/L) | 146.37 ± 137.82 | 58.78 ± 44.27 | 114.47 ± 69.14 | 260.93 ± 160.72 | 20.22 ± 10.73 |
| AST (U/L) | 146.92 ± 128.48 | 62.30 ± 39.51 | 120.97 ± 75.56 | 252.55 ± 153.51 | 25.61 ± 10.06 |
| Albumin | 34.05 ± 8.92 | 41.11 ± 4.87 | 35.90 ± 7.75 | 25.55 ± 5.57 | 42.17 ± 4.95 |
| (g/L) | | | | | |
| prothrombin | 16.19 ± 5.49 | 11.5 ± 1.32 | 14.91 ± 2.57 | 21.84 ± 5.25 | 10.86 ± 1.33 |
| time (s) | | | | | |

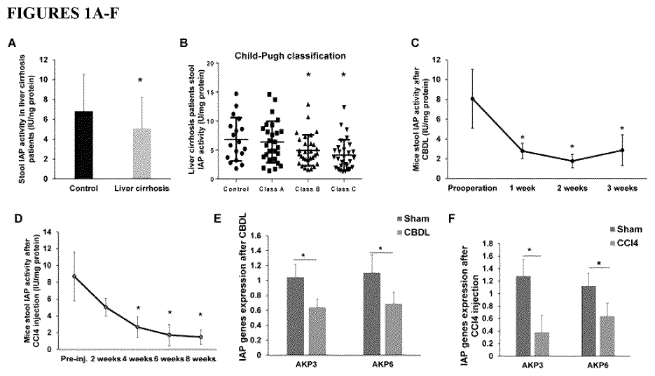

Compared to patients without cirrhosis, patients with cirrhosis had significantly lower fecal IAP activity (p<0.05, FIG. 1A). When comparing IAP activity by Child-Pugh grades, a decreasing trend was seen as liver function worsened. Although there was no difference in stool IAP activity between control patients and patients with class A liver function, control samples had a significantly higher IAP activity when compared with Child-Pugh B and C patients (p<0.05, FIG. 1).

### IAP Activity and Expression Decreased During Murine Liver Fibrosis

Given that liver fibrosis is associated with chronic intestinal inflammation, and endogenous IAP levels have been shown to be decreased in a variety of models of intestinal inflammation (Tuin et al., Gut. 2009; 58:379-87; Hamarneh et al., Dig Dis Sci. 2017; Molnar et al., Virchows Arch. 2012; 460:157-61; Kaliannan et al., Proc Natl Acad Sci USA. 2013; 110:7003-8; Gul et al., Appl Physiol Nutr Metab. 2017; 42:77-83), stool AP activity was measured using pNPP as a substrate for IAP. For mice that underwent CBDL, stool IAP activity significantly decreased from baseline preoperative stool IAP activity levels by 1 week postoperatively (p<0.05), and activity remained reduced when measured at both 2 weeks and 3 weeks after surgery (FIG. 1C). Similarly, in mice that received CCl4 injections, stool IAP activity began to decline from baseline after 2 weeks of injections. Stool IAP activity was significantly less than baseline at 4 weeks of injections and continued to decrease over the course of 8 weeks (p<0.05) (FIG. 1D). Compared to sham operated mice, both AKP-3 (duodenal IAP) and AKP-6 (global IAP) gene expression decreased in the duodenum of both the CBDL and CCl4 induced liver fibrosis models (FIG. 1E-F).

### IAP Regulates Gut Barrier in Murine Liver Fibrosis Models

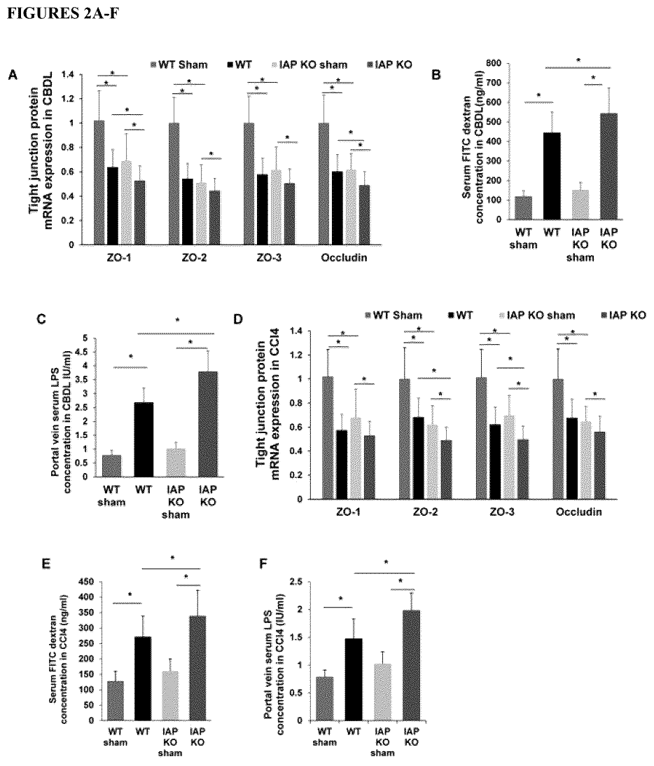

Impairment of the gut barrier is a component in the development of liver fibrosis. IAP has been shown to play a role in maintaining intestinal tight junction protein expression. The gut barrier function was tested in CBDL and CCl4 injected WT and IAP KO mice. Terminal ileum mRNA expression of ZO family and Occludin were significantly decreased in WT and IAP KO mice after both CBDL and CCl4 injection. At 3 weeks post-CBDL, ZO-2, ZO-3 and Occludin decreased more in WT mice than in IAP KO mice (FIG. 2A). At 8 weeks post CCl4 injection, ZO-1 and Occludin were more decreased in WT than in IAP KO mice (FIG. 2D).

In both WT and KO mice, serum FITC dextran was significantly higher post-CBDL than in sham operated mice, indicating impaired gut barrier function. IAP KO mice had worse gut barrier function than WT mice as evidenced by higher serum FITC dextran levels (p<0.05) (FIG. 2B). Terminal ileum ZO-1 and Occludin expression significantly decreased after 3 weeks of CBDL, with lower levels in IAP KO mice than WT mice (p<0.05) (FIG. 2A). Portal vein serum LPS concentration was measured as the portal venous system represents the most proximal exit point for bacterial inflammatory mediators originating from the gut lumen, and is another marker of gut barrier function. Portal vein LPS was significantly higher in mice that underwent CBDL, and IAP KO mice had a significantly higher portal vein LPS concentration when compared to WT (FIG. 2C). CCl4-treated mice had a similar pattern to CBDL mice. All CCl4 injected mice had significantly increased serum FITC dextran and portal vein LPS concentrations, as well as decreased ileum tight junction gene expression when compared to vehicle treated mice after 8 weeks (FIG. 2E-F).

### Lack of IAP Results in a Severe CBDL and CCL4 Induced-Liver Fibrosis in Mice

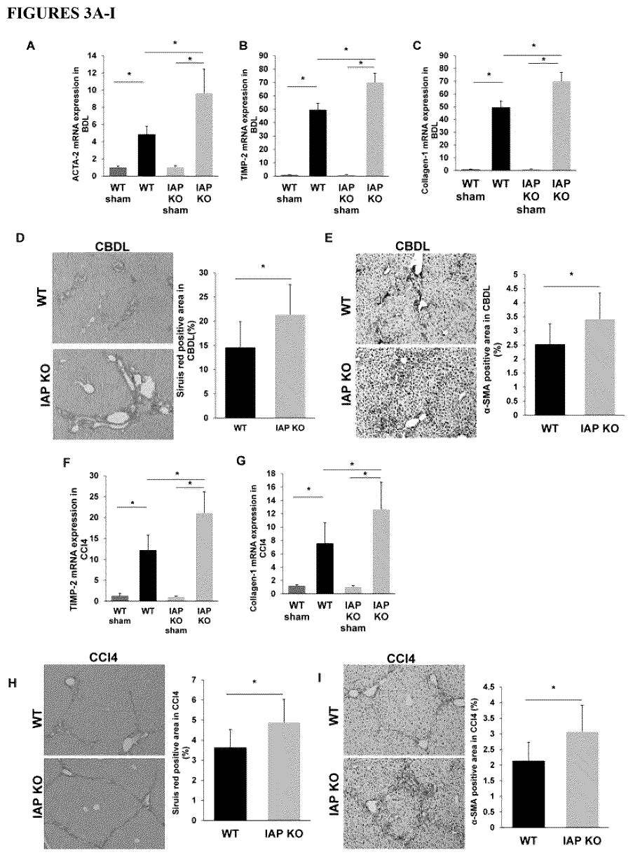

The gut lumen is a rich source of bacterial toxins, especially LPS, and these gut-derived mediators have been shown to participate in the pathogenesis of chronic liver inflammation/fibrosis (Seki et al., Nat Med. 2007; 13:1324-32). Thus, it was investigated whether the impaired gut barrier in IAP deficient mice resulted in worsened liver fibrosis. Liver fibrosis markers in both the CBDL and CCl4 models of liver injury was examined, and in both cases, a more severe damage and fibrosis was found with increased TIMP-2, Collagen-1, and ACTA-2 gene expressions. These genes associated with liver fibrosis also had higher expression in the IAP KO mice (FIG. 3A-C, F-G). Sirius red staining of sham operated mice showed minimal staining of collagen fiber, while staining was prominent after both CBDL and CCl4 injections. Collagen staining was more pronounced in the livers of IAP KO mice compared to WT mice (FIG. 3D, 3H). Similarly, α-SMA, another marker of fibrosis that is typically only found in the smooth muscle cells of the intrahepatic blood vessels under normal circumstances, was significantly increased in the livers of IAP KO mice compared to WT mice in both CBDL and CCl4 models (FIG. 3E, 3I).

### IAP Supplementation Protects the Gut Barrier in CBDL and CCL4 Induced-Liver Fibrosis in Mice

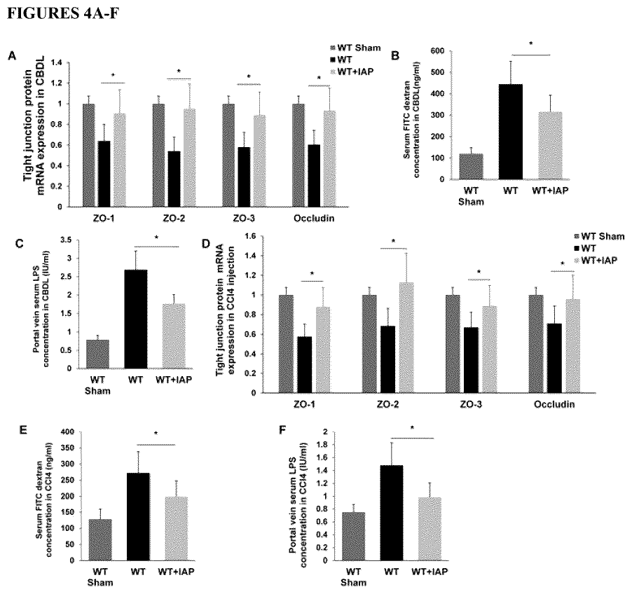

Next, it was tested whether supplementation with IAP in the drinking water could help to maintain the gut barrier function in murine models of liver fibrosis. As shown in FIGS. 4B and 4E, serum FITC labeled dextran concentrations increased in both WT mice and KO mice after CBDL and CCl4 injections. However, this increase was significantly attenuated by IAP supplementation. Furthermore, IAP supplementation also rescued the decrease in terminal ileum tight junction protein mRNA expression levels (FIG. 4A, 4D). In addition, portal vein LPS concentration was also significantly decreased by IAP supplementation in both models. (FIG. 4C, 4F).

### Oral IAP Attenuates Liver Fibrosis in Both CBDL and CCl4 Models

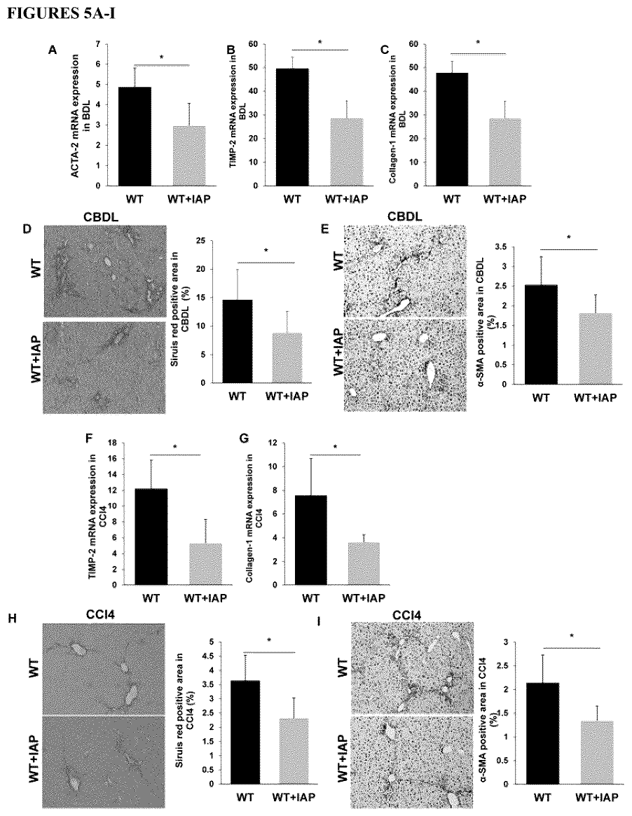

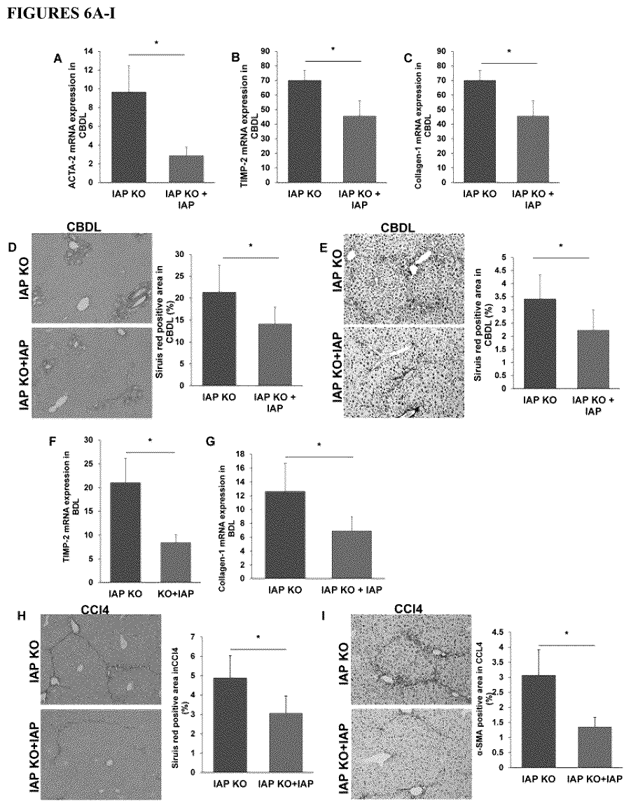

Translocation of gut derived LPS is a key driver of liver fibrosis, so it was tested whether oral IAP would maintain the gut barrier, detoxify luminal LPS, and attenuate the liver fibrosis induced by CBDL and CCl4 injury. After 3 weeks and 8 weeks of IAP supplementation in the CBDL and CCl4-treated mice, respectively, the data suggested that oral IAP significantly decreased expression of TIMP-1, Collagen-1, and ACTA-2 in both WT and KO mice (FIG. 5A-C, 5F-G; FIG. 6A-C, 6F-G).

On histologic evaluation, oral IAP resulted in a significant reduced Sirius red stained area for both WT and KO mice in both models compared to control WT and KO mice (FIG. 5D, 5H; FIG. 6D, 6H). Further, on immunohistochemistry, less staining of α-SMA was demonstrated in the livers of mice supplemented with IAP (FIG. 5E, 5I; FIG. 6E, 6I).

### IAP Rescues CBDL and CCL4-Induced Liver Fibrosis Dependent of TLR4 Pathway

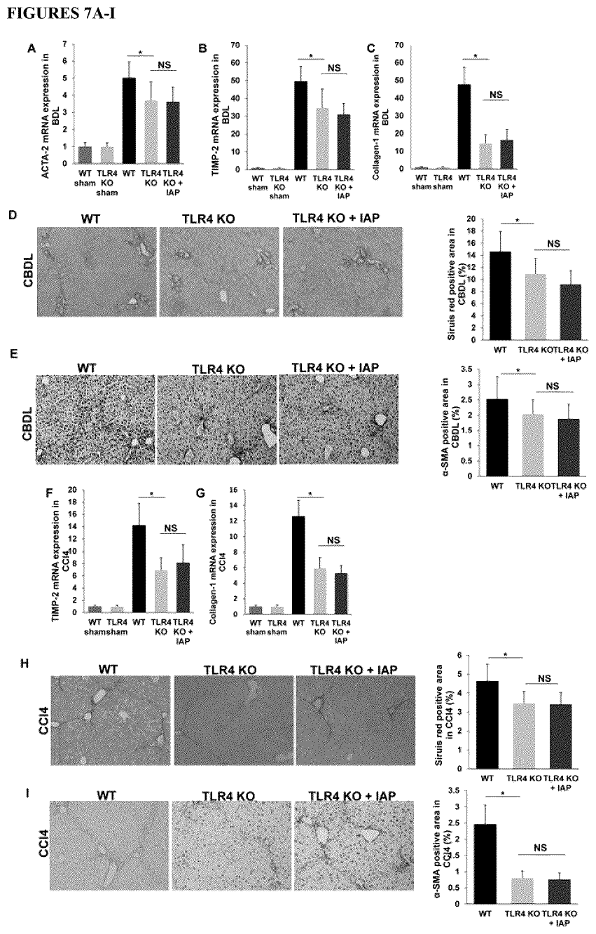

LPS induces liver fibrosis by translocating across the gut barrier, into the portal vein, where it then enters the liver, and activates the TLR4 pathway resulting in a variety of downstream signaling that lead to fibrosis (Seki et al., Nat Med. 2007; 13:1324-32). TLR4 KO mice have been shown to develop an attenuated degree of liver fibrosis in CBDL and CCl4 models compared to WT mice (Seki et al., Nat Med. 2007; 13:1324-32). In the present disclosure, mRNA expressions of ACTA-2, TIMP-2 and Collagen-1 were significantly lower in TLR4 mice compared to WT mice after both CBDL and CCl4 injection (FIG. 7A-C, 7F-G). Sirius red and α-SMA positive staining area were also significantly decreased in TLR4 KO mice compared to WT mice (FIG. 7D-E, 7H-I). This finding is consistent with previous results in both murine liver fibrosis models (Seki et al., Nat Med. 2007; 13:1324-32).

It is known that LPS is a substrate for IAP, and that IAP decreases luminal LPS (Bilski et al., Mediators Inflamm. 2017; 2017:9074601). It was examined whether the ability of IAP to prevent liver fibrosis is dependent on the TLR4 pathway. The liver fibrosis models were performed using TLR4 KO mice. While TLR4 KO mice had lower TIMP-2, ACTA-2, and Collagen-1 mRNA expression than WT mice after CBDL, they did not display a significant difference between IAP supplementation and control-treated mice (FIG. 7A-C). Similarly, Sirius red staining and α-SMA immunochemistry staining also did not show a difference between IAP or vehicle-treated mice in CBDL models (FIG. 7D-E). Similar results were shown in a CCl4 induced liver fibrosis model, as ACTA-2, TIMP-2 and Collagen-1 mRNA expression and Sirius red and α-SMA staining were similar between TLR4 KO and WT mice (FIG. 7F-H, 7I-J).

### Discussion

CCl4 and CBDL are two of the most commonly studied liver fibrosis animal models. These models begin with an insult to the liver, by either direct damage to hepatocytes (CCl4) or intrahepatic cholestasis (CBDL), followed by induction of key inflammatory pathways. Ultimately, this leads to the secretion of inflammatory cytokines, which in turn activate hepatic stellate cells via intrahepatic pathways thereby driving the development of fibrosis (Yanguas et al., Arch Toxicol. 2016; 90:1025-48). Importantly, the gastrointestinal tract and the liver are anatomically and physiologically connected by the portal venous system and biliary tree. An insult to the liver also manifests as changes to bile juice components and the gut microbiota, altering the gut epithelial barrier, further promoting translocation of pathogenic mediators into the portal venous system and liver (Ohtani et al., Hepatol Commun. 2019; 3:456-70).

The effects of mucosal-driven metabolites and luminal microbes on liver are considered important to the development of a variety of liver diseases (Ohtani et al., Hepatol Commun. 2019; 3:456-70). It has been demonstrated that intestinal barrier dysfunction and increased gut permeability are drivers of liver cirrhosis (Pijls et al., Liver Int. 2013; 33:1457-69; Assimakopoulos et al., Eur J Clin Invest. 2012; 42:439-46). In fact, gut barrier dysfunction not only occurs during the end stage of liver disease, but likely begins to take place in its early stages. Hepatic insults like excessive alcohol intake and high-fat diet have been shown to induce gut barrier breakdown (Wang Y et al., Mol Med Rep. 2014; 9:2352-6; Murphy et al., Curr Opin Clin Nutr Metab Care. 2015; 18:515-20). In murine models, expression of intestinal tight junction proteins is decreased as early as the first day after bile duct ligation or CCl4 injection (Fouts et al., J Hepatol. 2012; 56:1283-92).

Therefore, IAP supplementation was started 4 days in advance of the initiation of the hepatic insults, cholestasis in the case of CBDL and hepatoxicity in the case of CCl4. Translocation of intestinal bacteria and their by-products induces liver inflammation and plays an important role in liver fibrosis (Seki et al., Nat Med. 2007; 13:1324-32; Shi et al., Sci Rep. 2017; 7:40516). Breakdown of the protective gut barrier allows for more PAMPs such as LPS to pass through the intestine and become exposed to the liver. Despite the pathophysiologic importance of gut barrier alterations in the development of liver fibrosis, the mechanisms underlying the development of gut barrier dysfunction in this setting remain unclear.

IAP is well-known as a marker of enterocyte maturation and its activity has been negatively correlated with intestinal inflammation (Bilski et al., Mediators Inflamm. 2017; 2017:9074601). IAP activity has been shown to be decreased in diseases of chronic intestinal inflammation like inflammatory bowel disease (IBD), alcohol consumption, celiac disease, metabolic syndrome, and obesity (Tuin et al., Gut. 2009; 58:379-87; Hamarneh et al., Dig Dis Sci. 2017; Molnar et al., Virchows Arch. 2012; 460:157-61; Kaliannan et al., Proc Natl Acad Sci USA. 2013; 110:7003-8; Gul et al., Appl Physiol Nutr Metab. 2017; 42:77-83). Intestinal inflammation is also found in diseases such as nonalcoholic steatohepatitis (NASH), alcoholic liver disease (ALD), and other precursors to liver cirrhosis (Su et al., PloS one. 2018; 13:e0194867; Chen et al., Hepatology. 2015; 61:883-94). The present disclosure is the first to demonstrate that IAP activity is decreased both in human patients with liver cirrhosis and in murine models of liver fibrosis.

While gut barrier function relies on many different factors, the central component of the intestinal barrier are enterocytes that are tightly bound to adjacent cells by apical junctional proteins that include claudins, occludins, E-cadherins, desmosomes and junctional adhesion molecules (Tripathi et al., Nat Rev Gastroenterol Hepatol. 2018; 15:397-411; Turner et al., Nat Rev Immunol. 2009; 9:799-809). The integrity of the intestinal barrier becomes impaired when any of its key components are lost or become dysfunctional. This dysfunction can be induced by physiologic insults leading to gut inflammation and/or intestinal dysbiosis, such as high fat diet, alcohol consumption, lack of bile acids, IBD, etc. (Wang Y et al., Mol Med Rep. 2014; 9:2352-6; Murphy et al., Curr Opin Clin Nutr Metab Care. 2015; 18:515-20; Yang et al., Intensive Care Med. 2005; 31:709-17).

It has been shown before that IAP is essential for maintenance of the gut barrier in many disease models like systemic LPS exposure, fasting, alcohol-induced liver injury, and colitis (Liu et al. J Am Coll Surg. 2016; 222:1009-17; Hamarneh et al., Ann Surg. 2014; 260:706-14 discussion 14-5). When mice lack IAP, the gut barrier becomes significantly impaired and these mice are highly susceptible to physiologic insults that affect the gastrointestinal tract (Liu et al. J Am Coll Surg. 2016; 222:1009-17; Hamarneh et al., Ann Surg. 2014; 260:706-14 discussion 14-5).

The present disclosure suggests that gut barrier dysfunction is a step in development of liver fibrosis. When the gut barrier is damaged, intestinal bacteria-derived molecules, and even whole bacteria, will translocate from the gut to the liver via the portal vein, resulting in liver inflammation and injury, and ultimately liver fibrosis (Seki et al., Nat Med. 2007; 13:1324-32; Zhu et al., J Hepatol. 2012; 56:893-9). Further, when the liver becomes injured and cirrhosis develops, it also negatively impacts the gut barrier and increases bacteria translocation, leading to a vicious cycle of hepatic injury (Arab et al., Hepatol Int. 2018; 12:24-33).

The key function of IAP is its ability to detoxify a variety of pro-inflammatory mediators that exist within the gut lumen, including LPS, which is composed of hydrophilic polysaccharides within its core and O-antigen and a hydrophobic lipid A component. In recent years, LPS has been shown to be one of the key mediators linking the gut to the development of liver disease, as well as many other systematic diseases (Douhara et al., Mol Med Rep. 2015; 11:1693-700). IAP has been shown to remove a phosphate group from the lipid A moiety of LPS which leads to the amelioration of inflammatory activity of LPS (Miller et al., Nat Rev Microbiol. 2005; 3:36-46; Bates et al., Cell Host Microbe. 2007; 2:371-82; Poelstra et al., Am J Pathol. 1997; 151:1163-9). It has been shown that IAP knockout mice have an impaired ability to detoxify luminal LPS and appear to be more susceptible to gut-derived inflammatory conditions (Ramasamy et al., Inflamm Bowel Dis. 2011; 17:532-42; Kaliannan et al., Proc Natl Acad Sci USA. 2013; 110:7003-8). This is likely exacerbated in the scenario of the vicious cycle between gut and liver injury. Supplemental IAP or recombinant alkaline phosphatase has been shown to prevent alcohol-induced hepatosteatosis and acute on chronic liver failure (Hamarneh et al., Dig Dis Sci. 2017; Engelmann et al., Sci Rep. 2020; 10:389).

The present disclosure provides, inter alia, evidence that enteral supplementation of IAP in drinking water reduces the concentration of portal serum LPS that is derived from the gut lumen. Without being bound by theory, IAP may decrease the amount of active LPS that is translocated from the gut into the liver. This action may further attenuate the vicious inflammatory cycle and the development of liver fibrosis. The fact that IAP supplementation was not able to attenuate liver fibrosis in TLR4 KO mice confirms the important role of LPS in this setting.

It has been shown that targeting gram negative bacteria and LPS by long-term administration of rifaximin, polymyxin or norfloxacin may rescue liver fibrosis (Zhu et al., J Hepatol. 2012; 56:893-9; Douhara et al., Mol Med Rep. 2015; 11:1693-700; Gomez-Hurtado et al., Liver Int. 2018; 38:295-302). However, chronic antibiotic therapy is inherently associated with many negative side effects. In another study, the probiotic amino acid glutamine was found to protect the gut barrier and alleviate liver fibrosis in murine models (Shrestha et al., Food Chem Toxicol. 2016; 93:129-37). The results from the study put forward another novel therapy that may potentially prevent liver disease from progressing to liver fibrosis and cirrhosis. Enteral IAP has been used in one human study with intraduodenal administration for 7 days in patients with severe Ulcerative Colitis (Lukas et al., Inflamm Bowel Dis. 2010; 16:1180-6). In this limited single arm trial, no safety issues, adverse events, or side effects were reported. Given that IAP is an endogenously produced enzyme, it is not surprising that it could be safely administered to patients with few if any detrimental effects (Lukas et al., Inflamm Bowel Dis. 2010; 16:1180-6). Although IAP is partially degraded in the stomach, oral IAP in the drinking water is a very easy route of administration and it has been previously shown that this method of supplementation is effective to increase intestinal luminal concentration of IAP (Kuhn et al., JCI insight. 2020 5; Malo et al., Gut. 2010; 59:1476-84).

Although IAP functions both to directly maintain the gut barrier and also to detoxify bacterial ligands such as LPS, it is not clear which function of IAP plays a more important role in its ability to attenuate liver fibrosis. The mechanistic question was examined by using the TLR4 KO mice. The finding shows that the TLR4 KO mice did not have the same degree of improvement as wild type mice, suggests that this pathway is important to IAP action. With increased exposure of TLR4 to LPS and/or increased expression or sensitivity of TLR4, there is an inappropriate immune response which induces a chronic inflammatory liver disease (Vaure et al., Front Immunol. 2014; 5:316; Pimentel-Nunes et al., Expert Opin Ther Targets. 2010; 14:347-68). In recent years, many studies have provided evidence supporting the role of the LPS/TLR4 pathway in nonalcoholic fatty liver disease, alcoholic liver disease, acute liver failure, chronic hepatitis B and C, primary sclerosing cholangitis, primary biliary cirrhosis, liver fibrosis, and even hepatocellular carcinoma (Seki et al., Nat Med. 2007; 13:1324-32; Adachi et al., Gastroenterology. 1995; 108:218-24; Wang et al., Theranostics. 2020; 10:2714-26; Yang et al., Proc Natl Acad Sci USA. 1997; 94:2557-62; Isogawa et al., J Virol. 2005; 79:7269-72; Mao et al., Hepatology. 2005; 42:802-8; Karrar et al., Gastroenterology. 2007; 132:1504-14; Dapito et al., Cancer Cell. 2012; 21:504-16). In these experiments, IAP appears to rescue liver fibrosis only when TLR4 is present, suggesting (without wishing to be bound by theory) that IAP protects the liver from fibrosis by potentially de-activating LPS, an important ligand in the TLR4 pathway.

The data show that endogenous IAP is decreased during liver fibrosis. Without being bound by theory, the decrease in endogenous IAP during liver fibrosis may contribute to the gut barrier dysfunction, which results in an increased LPS translocation into the liver through the portal system, worsens liver damage, and creates a vicious cycle, ultimately leading to liver fibrosis. Oral IAP protects the gut barrier and further prevents the development of liver fibrosis via a TLR4-mediated mechanism. Thus, the data presented herein demonstrate that oral IAP represents a novel therapy to prevent the development of liver fibrosis by detoxifying luminal LPS and protecting the gut barrier function, in a TLR4-dependent fashion.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### REFERENCES

1. Ohtani N, Kawada N. Role of the Gut-Liver Axis in Liver Inflammation, Fibrosis, and Cancer: A Special Focus on the Gut Microbiota Relationship. Hepatol Commun. 2019; 3:456-70
2. Wang Y, Tong J, Chang B, Wang B, Zhang D, Wang B. Effects of alcohol on intestinal epithelial barrier permeability and expression of tight junction-associated proteins. Mol Med Rep. 2014; 9:2352-6
3. Murphy E A, Velazquez K T, Herbert K M. Influence of high-fat diet on gut microbiota: a driving force for chronic disease risk. Curr Opin Clin Nutr Metab Care. 2015; 18:515-20
4. Wiest R, Lawson M, Geuking M. Pathological bacterial translocation in liver cirrhosis. J Hepatol. 2014; 60:197-209
5. Seki E, De Minicis S, Osterreicher C H, Kluwe J, Osawa Y, Brenner D A. et al. TLR4 enhances TGF-β signaling and hepatic fibrosis. Nat Med. 2007; 13:1324-32
6. Bilski J, Mazur-Bialy A, Wojcik D, Zahradnik-Bilska J, Brzozowski B, Magierowski M. et al. The Role of Intestinal Alkaline Phosphatase in Inflammatory Disorders of Gastrointestinal Tract. Mediators Inflamm. 2017; 2017:9074601
7. Tuin A, Poelstra K, de Jager-Krikken A, Bok L, Raaben W, Velders M P. et al. Role of alkaline phosphatase in colitis in man and rats. Gut. 2009; 58:379-87
8. Ramasamy S, Nguyen D D, Eston M A, Alam S N, Moss A K, Ebrahimi F. et al. Intestinal alkaline phosphatase has beneficial effects in mouse models of chronic colitis. Inflamm Bowel Dis. 2011; 17:532-42
9. Hamarneh S R, Kim B M, Kaliannan K, Morrison S A, Tantillo T J, Tao Q. et al. Intestinal Alkaline Phosphatase Attenuates Alcohol-Induced Hepatosteatosis in Mice. Dig Dis Sci. 2017
10. Soares J B, Pimentel-Nunes P, Roncon-Albuquerque R, Leite-Moreira A. The role of lipopolysaccharide/toll-like receptor 4 signaling in chronic liver diseases. Hepatol Int. 2010; 4:659-72
11. Liu W, Hu D, Huo H, Zhang W, Adiliaghdam F, Morrison S. et al. Intestinal Alkaline Phosphatase Regulates Tight Junction Protein Levels. J Am Coll Surg. 2016; 222:1009-17
12. Kuhn F, Adiliaghdam F, Cavallaro P M, Hamarneh S R, Tsurumi A, Hoda R S. et al. Intestinal alkaline phosphatase targets the gut barrier to prevent aging. JCI insight. 2020 5
13. Narisawa S, Huang L, Iwasaki A, Hasegawa H, Alpers D H, Millan J L. Accelerated fat absorption in intestinal alkaline phosphatase knockout mice. Mol Cell Biol. 2003; 23:7525-30
14. Molnar K, Vannay A, Sziksz E, Banki N F, Gyorffy H, Arato A. et al. Decreased mucosal expression of intestinal alkaline phosphatase in children with coeliac disease. Virchows Arch. 2012; 460:157-61
15. Kaliannan K, Hamarneh S R, Economopoulos K P, Nasrin Alam S, Moaven O, Patel P. et al. Intestinal alkaline phosphatase prevents metabolic syndrome in mice. Proc Natl Acad Sci USA. 2013; 110:7003-8
16. Gul S S, Hamilton A R, Munoz A R, Phupitakphol T, Liu W, Hyoju S K. et al. Inhibition of the gut enzyme intestinal alkaline phosphatase may explain how aspartame promotes glucose intolerance and obesity in mice. Appl Physiol Nutr Metab. 2017; 42:77-83
17. Yanguas S C, Cogliati B, Willebrords J, Maes M, Colle I, van den Bossche B. et al. Experimental models of liver fibrosis. Arch Toxicol. 2016; 90:1025-48
18. Pijls K E, Jonkers D M, Elamin E E, Masclee A A, Koek G H. Intestinal epithelial barrier function in liver cirrhosis: an extensive review of the literature. Liver Int. 2013; 33:1457-69
19. Assimakopoulos S F, Tsamandas A C, Tsiaoussis G I, Karatza E, Triantos C, Vagianos C E. et al. Altered intestinal tight junctions' expression in patients with liver cirrhosis: a pathogenetic mechanism of intestinal hyperpermeability. Eur J Clin Invest. 2012; 42:439-46
20. Fouts D E, Torralba M, Nelson K E, Brenner D A, Schnabl B. Bacterial translocation and changes in the intestinal microbiome in mouse models of liver disease. J Hepatol. 2012; 56:1283-92
21. Shi H, Lv L, Cao H, Lu H, Zhou N, Yang J. et al. Bacterial translocation aggravates CCl4-induced liver cirrhosis by regulating CD4(+) T cells in rats. Sci Rep. 2017; 7:40516
22. Su Y B, Li T H, Huang C C, Tsai H C, Huang S F, Hsieh Y C. et al. Chronic calcitriol supplementation improves the inflammatory profiles of circulating monocytes and the associated intestinal/adipose tissue alteration in a diet-induced steatohepatitis rat model. PloS one. 2018; 13:e0194867
23. Chen P, Starkel P, Turner J R, Ho S B, Schnabl B. Dysbiosis-induced intestinal inflammation activates tumor necrosis factor receptor I and mediates alcoholic liver disease in mice. Hepatology. 2015; 61:883-94
24. Tripathi A, Debelius J, Brenner D A, Karin M, Loomba R, Schnabl B. et al. The gut-liver axis and the intersection with the microbiome. Nat Rev Gastroenterol Hepatol. 2018; 15:397-411
25. Turner J R. Intestinal mucosal barrier function in health and disease. Nat Rev Immunol. 2009; 9:799-809
26. Yang R, Harada T, Li J, Uchiyama T, Han Y, Englert J A. et al. Bile modulates intestinal epithelial barrier function via an extracellular signal related kinase 1/2 dependent mechanism. Intensive Care Med. 2005; 31:709-17
27. Hamarneh S R, Mohamed M M, Economopoulos K P, Morrison S A, Phupitakphol T, Tantillo T J. et al. A novel approach to maintain gut mucosal integrity using an oral enzyme supplement. Ann Surg. 2014; 260:706-14 discussion 14-5
28. Zhu Q, Zou L, Jagavelu K, Simonetto D A, Huebert R C, Jiang Z D. et al. Intestinal decontamination inhibits TLR4 dependent fibronectin-mediated cross-talk between stellate cells and endothelial cells in liver fibrosis in mice. J Hepatol. 2012; 56:893-9
29. Arab J P, Martin-Mateos R M, Shah V H. Gut-liver axis, cirrhosis and portal hypertension: the chicken and the egg. Hepatol Int. 2018; 12:24-33
30. Douhara A, Moriya K, Yoshiji H, Noguchi R, Namisaki T, Kitade M. et al. Reduction of endotoxin attenuates liver fibrosis through suppression of hepatic stellate cell activation and remission of intestinal permeability in a rat non-alcoholic steatohepatitis model. Mol Med Rep. 2015; 11:1693-700
31. Miller S I, Ernst R K, Bader M W. LPS, TLR4 and infectious disease diversity. Nat Rev Microbiol. 2005; 3:36-46
32. Bates J M, Akerlund J, Mittge E, Guillemin K. Intestinal alkaline phosphatase detoxifies lipopolysaccharide and prevents inflammation in zebrafish in response to the gut microbiota. Cell Host Microbe. 2007; 2:371-82
33. Poelstra K, Bakker W W, Klok P A, Kamps J A, Hardonk M J, Meijer D K. Dephosphorylation of endotoxin by alkaline phosphatase in vivo. Am J Pathol. 1997; 151:1163-9
34. Engelmann C, Adebayo D, Oria M, De Chiara F, Novelli S, Habtesion A. et al. Recombinant Alkaline Phosphatase Prevents Acute on Chronic Liver Failure. Sci Rep. 2020; 10:389
35. Gomez-Hurtado I, Gimenez P, Garcia I, Zapater P, Frances R, Gonzalez-Navajas J M. et al. Norfloxacin is more effective than Rifaximin in avoiding bacterial translocation in an animal model of cirrhosis. Liver Int. 2018; 38:295-302
36. Shrestha N, Chand L, Han M K, Lee S O, Kim C Y, Jeong Y J. Glutamine inhibits CCl4 induced liver fibrosis in mice and TGF-beta1 mediated epithelial-mesenchymal transition in mouse hepatocytes. Food Chem Toxicol. 2016; 93:129-37
37. Lukas M, Drastich P, Konecny M, Gionchetti P, Urban O, Cantoni F. et al. Exogenous alkaline phosphatase for the treatment of patients with moderate to severe ulcerative colitis. Inflamm Bowel Dis. 2010; 16:1180-6
38. Malo M S, Alam S N, Mostafa G, Zeller S J, Johnson P V, Mohammad N. et al. Intestinal alkaline phosphatase preserves the normal homeostasis of gut microbiota. Gut. 2010; 59:1476-84
39. Vaure C, Liu Y. A comparative review of toll-like receptor 4 expression and functionality in different animal species. Front Immunol. 2014; 5:316
40. Pimentel-Nunes P, Soares J B, Roncon-Albuquerque R Jr, Dinis-Ribeiro M, Leite-Moreira A F. Toll-like receptors as therapeutic targets in gastrointestinal diseases. Expert Opin Ther Targets. 2010; 14:347-68
41. Adachi Y, Moore L E, Bradford B U, Gao W, Thurman R G. Antibiotics prevent liver injury in rats following long-term exposure to ethanol. Gastroenterology. 1995; 108:218-24
42. Wang F, Gong S, Wang T, Li L, Luo H, Wang J. et al. Soyasaponin II protects against acute liver failure through diminishing YB-1 phosphorylation and Nlrp3-inflammasome priming in mice. Theranostics. 2020; 10:2714-26
43. Yang S Q, Lin H Z, Lane M D, Clemens M, Diehl A M. Obesity increases sensitivity to endotoxin liver injury: implications for the pathogenesis of steatohepatitis. Proc Natl Acad Sci USA. 1997; 94:2557-62
44. Isogawa M, Robek M D, Furuichi Y, Chisari F V. Toll-like receptor signaling inhibits hepatitis B virus replication in vivo. J Virol. 2005; 79:7269-72
45. Mao T K, Lian Z X, Selmi C, Ichiki Y, Ashwood P, Ansari A A. et al. Altered monocyte responses to defined TLR ligands in patients with primary biliary cirrhosis. Hepatology. 2005; 42:802-8
46. Karrar A, Broome U, Sodergren T, Jaksch M, Bergquist A, Bjornstedt M. et al. Biliary epithelial cell antibodies link adaptive and innate immune responses in primary sclerosing cholangitis. Gastroenterology. 2007; 132:1504-14
47. Dapito D H, Mencin A, Gwak G Y, Pradere J P, Jang M K, Mederacke I. et al. Promotion of hepatocellular carcinoma by the intestinal microbiota and TLR4. Cancer Cell. 2012; 21:504-16

1. A composition comprising an alkaline phosphatase (AP)-based agent for use in a method of treating or inhibiting liver fibrosis secondary to a liver disease or disorder characterized by hepatotoxicity in a subject in need thereof, the method comprising administering an effective amount of the alkaline phosphatase (AP)-based agent to the subject,
wherein the liver disease or disorder characterized by hepatotoxicity is selected from nonalcoholic fatty liver disease (NAFLD), nonalcoholic steatohepatitis (NASH), and acute-on-chronic liver failure (ACLF).

2. (canceled)

3. The composition of claim 1, wherein the AP-based agent is intestinal alkaline phosphatase (IAP).

4. The composition of claim 3, wherein the IAP is bovine IAP (bIAP).

5. The composition of claim 4, wherein the bIAP comprises an amino sequence having at least about 90% sequence identity to one of SEQ ID NO: 1 to SEQ ID NO: 11.

6. (canceled)

7. A method of treating or inhibiting liver fibrosis secondary to a liver disease or disorder characterized by hepatotoxicity in a subject in need thereof, the method comprising administering an effective amount of an alkaline phosphatase (AP)-based agent to the subject, wherein the liver disease or disorder characterized by hepatotoxicity is selected from nonalcoholic fatty liver disease (NAFLD), nonalcoholic steatohepatitis (NASH), and acute-on-chronic liver failure (ACLF).

8. A method of treating or inhibiting liver fibrosis in a subject in need thereof, the method comprising administering an effective amount of an alkaline phosphatase (AP)-based agent to the subject, wherein the liver fibrosis has a non-biliary etiology.

9. The method of claim 7, wherein the liver fibrosis is not characterized by cholestasis.

10.-15. (canceled)

16. The method of claim 7, wherein the method prevents or mitigates the development of one or more of cirrhosis, end-stage liver disease, or hepatocellular carcinoma.

17. The method of claim 7, wherein the method prevents or mitigates the development of one or more of periportal fibrosis, bridging fibrosis, or cirrhosis.

18. The method of claim 7, wherein the subject:
is afflicted by one or more of insulin resistance, pre-diabetes, type 2 diabetes mellitus, and obesity, or
does not regularly consume alcohol.

19. The method of claim 7, wherein the subject has hepatocellular ballooning.

20. The method of claim 7, wherein the administration results in a decrease or lack of increase in expression or activity of one or more of tissue inhibitor of metalloproteinases-1 (TIMP-1), collagen-1, and smooth muscle alpha α-2 actin (ACTA-2).

21. The method of claim 7, wherein the administration results in a decrease or lack of increase in expression or activity of Smooth muscle alpha-actin (α-SMA).

22. The method of claim 7, wherein the AP-based agent is intestinal alkaline phosphatase (IAP).

23. The method of claim 22, wherein the IAP is bovine IAP (bIAP).

24. The method of claim 23, wherein the bIAP comprises an amino sequence having at least about 90% sequence identity to one of SEQ ID NO: 1 to SEQ ID NO: 11.

25. The method of claim 24, wherein the bIAP comprises an amino sequence having at least about 97% sequence identity to one of SEQ ID NO: 11 or one of SEQ ID NO: 1 to SEQ ID NO: 10.

26. The method of claim 7, wherein the AP-based agent is administered enterally or parenterally.

27. The method of claim 26, wherein the enteral administration is oral administration.

28. The method of claim 7, wherein the subject is a human patient.
